SEQUENCE LISTING (3 sequences)

SEQ ID NO: 1 - DNA, 462 bp, Homo Sapiens
  CDS (1)...(462): Coding sequence of human IL-2
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 2 - DNA, 462 bp, Artificial Sequence
  CDS (1)...(462): Optimized coding sequence of human IL-2
atgtaccgcatgcagctgctgagctgcatcgccctgagcctggccctggtgaccaacagcgcccccaccagcagcagcaccaagaagacccagctgcagctggagcacctgctgctggacctgcagatgatcctgaacggcatcaacaactacaagaaccccaagctgacccgcatgctgaccttcaagttctacatgcccaagaaggccaccgagctgaagcacctgcagtgcctggaggaggagctgaagcccctggaggaggtgctgaacctggcccagagcaagaacttccacctgcgcccccgcgacctgatcagcaacatcaacgtgatcgtgctggagctgaagggcagcgagaccaccttcatgtgcgagtacgccgacgagaccgccaccatcgtggagttcctgaaccgctggatcaccttctgccagagcatcatcagcaccctgacctga

SEQ ID NO: 3 - protein, 153 aa, Homo Sapiens
  Amino acid sequence of human IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

(19) United States
(12) Patent Application Publication
(10) Pub. No.: US 2002/0058795 A1
(43) Pub. Date: May 16, 2002

(54) HYDROPHOBIC GLYCOSYLAMINE DERIVATIVES, COMPOSITIONS, AND METHODS FOR USE

(76) Inventors: RUSS J. MUMPER, THE WOODLANDS, TX (US); FRANK TAGLIAFERRI, HOUSTON, TX (US)

Correspondence Address: LYON & LYON LLP/ VALENTIS INC., 633 WEST FIFTH STREET, SUITE 4700, LOS ANGELES, CA, 90071-2066, US

(21) Appl. No.: 09/149,721

(22) Filed: Sep. 8, 1998

Related U.S. Application Data
(60) Provisional application No. 60/058,259, filed on Sep. 8, 1997.

(51) Int. Cl. C07H 17/08; C07H 15/00; C07H 17/00; C07H 17/02

(52) U.S. Cl. 536/7.2

(57) ABSTRACT

The invention relates in part to hydrophobic glycosylamine derivatives, methods for synthesizing hydrophobic derivatives, compositions comprising these derivatives, and methods for delivering macromolecules to cells by administering these compositions.

### RELATED APPLICATION

The present application claims priority to U.S. Ser. No. 60/058,259, filed Sep. 8, 1997, entitled HYDROPHOBIC GLYCOSYLAMINE DERIVATIVES, COMPOSITIONS, AND METHODS FOR USE, by Mumper et al. (Lyon & Lyon Docket No. 225/299), which is incorporated herein by reference in its entirety, including any drawings.

### FIELD OF THE INVENTION

The invention relates generally to the delivery of macromolecules to cells. In particular, the invention relates in part to the delivery of nucleic acid plasmid molecules to cells and the transcription of ribonucleic acid molecules and translation of polypeptides from these plasmid molecules.

### BACKGROUND OF THE INVENTION

The following discussion of the background of the invention is merely provided to aid the reader in understanding the invention and is not admitted to describe or constitute prior art to the present invention.

The transfer of genes into cells of various origins is an important technique for biological research. Gene transfer can facilitate straightforward studies for the function and regulation of genes and proteins in cells cultured in vitro as well as in cells of multi-cellular organisms. Besides being a powerful research tool, gene transfer also has important economic implications through the genetic engineering of micro-organisms, plants, and animals, for the production of proteins as well as for crop and livestock improvement. Behr, 1994, Bioconjugate Chem. 5: 382-389.

There has been an increasing interest in gene transfer techniques in view of recent advances in gene therapy. Gene therapy is the treatment of diseased organisms by replacing or modifying damaged genes which lead to diseased states. Many ongoing clinical protocols make use of recombinant retroviruses which generally are by far the most efficient vehicles to integrate foreign DNA into the genome of dividing cells. Adenoviral factors have also been shown recently to efficiently transfect a large variety of post-mitotic cells. These and other currently developed biological vectors (HSV, AAV), however, raise non-assessable long-term risks and have a limited capacity to carry foreign genetic material. Id.

Current non-viral approaches to gene transfer require that a potential therapeutic gene be cloned into plasmids. Large quantities of a bacterial host harboring the plasmid may be fermented and the plasmid DNA may be purified for subsequent use. Current human clinical trials using plasmids utilize this approach. Recombinant DNA Advisory Committee Data Management Report, December, 1994, Human Gene Therapy 6: 535-548. Studies normally focus on the therapeutic gene and the elements that control its expression in the patient when designing and constructing gene therapy plasmids. Generally, therapeutic genes and regulatory elements are simply inserted into existing cloning vectors that are convenient and readily available. For example, the gene encoding interleukin-2 can be cloned into plasmid vectors appropriate for gene therapy applications.

Interleukin-2 (IL-2) is involved in stimulating the proliferation of helper T cells, in particular TH1 cells by an autocrine mechanism. Secretion of IL-2 can also stimulate the proliferation of other activated helper T cells and cytotoxic T cells.

Based on these and other responses to IL-2, attempts have been made to use IL-2 in anti-tumor therapy. IL-2 polypeptides can be isolated from stimulated TH1 cells or produced from a recombinant IL-2 gene. Such a recombinant gene is described, for example, in Taniguchi et al., U.S. Pat. No. 4,738,927, along with cells containing the recombinant gene. However, administration of high doses of IL-2 polypeptide can result in significant toxicity-related side effects, such as fever, fluid retention, and vascular leak syndrome. In addition, administration of polypeptide must be repeated at short intervals due to a short half-life for the injected polypeptide.

Once an appropriate gene and plasmid-DNA vector are identified for gene therapy techniques, the DNA plasmid must be formulated into a composition appropriate for gene transfer. One of the first compounds used to formulate a composition of plasmid DNA for gene transfer was DOTMA ((dioleoyloxypropyl)trimethylammonium bromide). This cationic lipid can form compositions that can efficiently transfer DNA and RNA into eukaryotic cell lines. Felgner et al., 1987, Proc. Natl. Acad. Sci. U.S.A. 84: 7413-7417 and Malone et al., 1989, Proc. Natl. Acad. Sci. U.S.A. 86:, 6077-6081. DOTMA has been commercialized (Lipofectin, Gibco-BRL) as a one to one mixture with DOPE (dioleoylphosphatidyl ethanolamine). This mixture has been used to transfect a variety of animal and plant eukaryotic cells. Other cationic lipids have been synthesized in addition to DOTMA, such as DOTB and DOTAP dioleylesters. Levintest et al. 1990, Biochem. Biophys. Acta 1023: 124-132.

Other examples of cationic lipids are DOGS and DPPES. DOGS and DPPES have been shown to transfect established cell lines and primary neuronal cultures more efficiently than calcium phosphate by at least two orders of magnitude. Behr, 1986, Proc. Natl. Acad. Sci. U.S.A. 86: 6982-6986.

For the formation of liposome compositions, positively charged methyl-2-amino-6-palmitoyl-D-glycosides (PGLNs) have been synthesized as gene transfer components. Aoki et al., 1995, Int. J. Pharm. 115: 183-191. These PGLNs could be derivatives of glucose, lactose, and mannose. In addition, synthetic glycosylamine diesters have been synthesized for the transfer of superoxide dismutase into cells. Miyajima et al., 1993, Chem. Pharm. Bull. 41: 1889-1894.

### SUMMARY

The invention relates in part to hydrophobic glycosylamine derivatives, methods of synthesizing hydrophobic derivatives, compositions comprising these hydrophobic derivatives, and methods for delivering macromolecules to cells by administering these compositions. The compounds, compositions, and methods of the invention are particularly useful for gene therapy applications. Specifically, the invention can be applied towards treating cancer. For example, a polypeptide, such as interleukin-2, may be expressed from a DNA plasmid delivered to cells by the methods described herein. The interleukin-2 gene product can significantly reduce the growth rate of tumors in mammals.

The invention provides a number of biodegradable cationic glycolipids, also named hydrophobic glycosylamine derivatives (HGDs), which can deliver DNA plasmids into cells. The HGDs of the present invention are based upon an amino-sugar backbone which has been modified by the addition of a lipophilic moiety such as, but not limited to, alkyl or acyl chains. These modifications may take place at the 1, 3, 4, and/or 6 positions of any number of 2-deoxy-2-amino sugars (i.e., glucosamine or mannosamine). Alternatively, a cationic functionality may be introduced at a position other than the 2-position (i.e., 6-amino-6-deoxy glucose) and lipophilic modification could then be made at any or all of the non-cation containing positions. Furthermore, two or more cationic functionalities may be introduced onto a carbohydrate backbone in order to provide additional sites for positive charge. Such species could then be modified in an analogous manner to mono-amine carbohydrates.

The HGDs of the invention can deliver DNA molecules into cells without the step of pre-forming liposomes. The majority of commercially available cationic lipids transfer DNA into cells via a DNA complex formed by (1) preparing liposomes from the cationic lipid, preferably in conjunction with of a co-lipid such as DOPE, and (2) forming a DNA complex by adding DNA to the liposome suspension. These steps, as well as the properties of multiple cationic lipids, are defined in Felgner and Ringold, 1989, “Cationic Liposome-Mediated Transfection,” Nature 337: 387-388 and Gao and Huang, 1995, “Cationic Liposome-Mediated Gene Transfer,” Gene Therapy 2: 710-722.

Instead, the HGDs of the invention can transfer DNA molecules into cells via condensed or non-condensed DNA/HGD complexes prepared in one step by simply mixing the HGDs with DNA. The degree to which a DNA complex is condensed can depend upon the charge ratio of the DNA/HGD complex. The HGDs of the invention may or may not form micelles or liposomes prior to addition of DNA. As shown below, non-condensed DNA/HGD complexes can efficiently deliver DNA of therapeutic value into cancerous tumors.

I. Hydrophobic Glycosylamine Derivatives of the Invention

In a first aspect, the invention features a compound comprising a glycosyl moiety having a nitrogen-based substituent linked to a carbon atom within the glycosyl moiety, where the nitrogen-based substituent is selected from the group consisting of —NH₂, —N⁺(CH₃)₃, —(CH₂)ₙ—N(R₁₀)₃, and —NH—C(N+H₂)—NH₂, and where substituents linked to other carbon atoms within the glycosyl moiety are selected from the group consisting of hydrogen, -alkyl, —O— alkyl, —O—C(O)-alkyl,

—O—CH₂—CH₂(O—C(O)—R₆)—CH₂ (O—C (O)—R₇) —O—CH₂-CH₂ (OR₆)—CH₂ (OR₇) , —O—CH₂—CH₂ (R₆)—CH₂ (R₇), —O—(CH₂)ₘ-cholesterol, polyethylene glycol, —O—(CH₂)ₙ—N(R₈)₃, —NH₂, —N⁺(CH₃)₃, —(CH₂)ₙ—N (R₉)₃, and —(CH₂)—OR₁₀ where R₆, R₇, R₈, R₉, and R₁₀ are independently selected from the group consisting of hydrogen, methyl, and alkyl, and where m is selected from the group consisting of 0, 1, 2, 3, 4, and 5, and where n is selected from the group consisting of 1, 2, 3, 4, and 5.

The term “hydrophobic glycosylamine derivative” as used herein refers to a glycosyl moiety having a nitrogen-based substituent attached to one of the carbon atoms of the glycosyl moiety and having at least one substituent attached to another carbon atom of the glycosyl moiety that comprises a hydrophobic moiety. These hydrophobic glycosylamine derivatives can be derived from naturally occurring amino sugars. These naturally occurring amino sugars can have structures set forth in the following formulas:

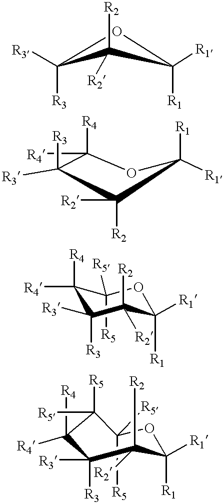

R₁, R₁′, R₂, R₂′, R₃, R₃′, R₄, R₄′, R₅, and R₅′ can be independently selected from the group consisting of any of the chemical moieties described in the preceding paragraph, provided that at least one of these R-substituents is a nitrogen-based moiety. Examples of amino sugars are 2-amino-2-deoxy-galactose, 2-amino-2-deoxy-glucose, 2-amino-2-deoxy-mannose. Glycosyl derivatives can also relate to glyceraldehyde, erythrose, threose, ribose, arabinose, xylose, lyxose, allose, altrose, glucose, mannose, glucose, idose, galactose, talose, dihydroxyacetone, erythylose, ribulose, xylulose, psicose, fructose, sorbose, and tagatose derivatives. These examples are not meant to be limiting; glycosylamine derivatives of the invention can relate to any known glycosyl moiety.

The term “nitrogen-based” as used herein refers to any chemical moiety that comprises a nitrogen atom or ion thereof. Preferably, the nitrogen atom is attached to a carbon atom within the glycosyl moiety. Examples of nitrogen-based compounds are set forth above.

The term “hydrophobic” as used herein refers to a chemical moiety or moieties that partition into non-polar environments with a higher probability than they partition into polar environments. Hydrophobic moieties can be attached to peptides of the invention to confer a lipophilic character to the peptides. The hydrophobic character of a molecule can be determined by partitioning the molecule into a mixture of water and octanol, which is a technique well known to a person of ordinary skill in the art. Thus, a hydrophobic molecule will partition into octanol with a higher probability than it will partition into water.

The term “linked” as used herein refers to two chemical moieties being associated with one another by a covalent chemical bond. This covalent bond may exist as a single, double, or triple bond.

The term “within the glycosyl moiety” as used herein refers to atoms that comprise the glycosyl moiety backbone. These atoms can be selected from nitrogen and sulfur, preferably oxygen, and most preferably carbon. The glycosyl moiety backbone refers to the backbone atoms within a glycosyl moiety that exists in a straight-chain form or in a cyclized form.

Other terms are defined with respect to the aspect of the invention described directly below.

In another aspect, the invention features a compound having a structure set forth in formula I:

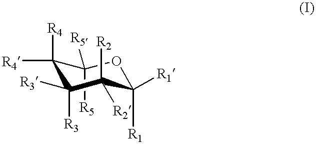

where (a) R₁ and R₁′ are independently selected from the group consisting of hydrogen, —OH, —OCH₃, -alkyl, —O— alkyl, —O—C(O)-alkyl, —O—CH₂—CH₂ (O—C (O)—R₆)—CH₂ (O—C (O)—R₇), —O—CH₂—CH₂ (OR₆) —CH₂ (OR₇) , —O—CH₂—CH₂ (R₆)—CH₂ (R₇), —O—(CH₂)ₘ-cholesterol, polyethylene glycol, —O—(CH₂)ₙ—N(R₈)₃, —NH₂, —N⁺(CH₃)₃, and —(CH₂)ₙ—N(R₉)₃, where R₆, R₇, R₈, and R₉ are independently selected from the group consisting of hydrogen, methyl, and alkyl, and where m is selected from the group consisting of 0, 1, 2, 3, 4, and 5, and where n is selected from the group consisting of 1, 2, 3, 4, and 5; (b) R₂ and R₂′ are independently selected from the group consisting of hydrogen, —NH₂, —N⁺(CH₃)₃, —(CH₂)ₙ— N(R₁₁)₃, and

—NH—C(N⁺H₂)—NH₂, wherein R₁₁ is selected from the group consisting of hydrogen, methyl, and alkyl; and (c) R₃, R₃′, R₄, R₄′, R₅, and R₅′ are independently selected from the group consisting of hydrogen —OH, —OCH₃, -alkyl, —O— alkyl, —O—C(O)-alkyl, —O—CH₂—CH₂ (O—C (O)—R₆) —CH₂ (O—C (O)—R₇), —O—CH₂—CH₂ (OR₆) —CH₂ (OR₇), —O—CH₂—CH₂ (R₆) —CH₂ (R₇), —O—(CH₂)ₘ-cholesterol, polyethylene glycol, —O—(CH₂)ₙ—N (R₈)₃, —NH₂, —N⁺(CH₃)₃, and —(CH₂)ₙ—N (R₉)₃, where R₆, R₇, R₈, and R₉are independently selected from the group consisting of hydrogen, methyl, and alkyl, and where m is selected from the group consisting of 0, 1, 2, 3, 4, and 5, and where n is selected from the group consisting of 1, 2, 3, 4, and 5, provided that R₅′ is not —CH₂—O—C(O)—(CH₂)₁₄CH₃ when R₃′ and R4′ are —OH and R₂′ is —NH₂ and R₁ is —OCH₃, and provided that R₅′ is not —CH₂—O—C(O)—(CH₂)ₚCH₃, where p is selected from the group consisting of 10, 12, 14, or 16, when R₃′ is identical to R₅′ and R₄′ is —OH and R₂ is —NH₂ and R₁′ is —OCH₃.

The term “alkyl” as used herein refers to a hydrocarbon molecule. An alkyl moiety of the invention preferably comprises 9 to 24 carbon atoms, more preferably comprises 12 to 20 carbon atoms, and most preferably comprises 14 to 18 carbon atoms. The alkyl moiety may have a formula of —(CH₂)ₙCH₃, where n is preferably 8 to 24, more preferably 10 to 20, and most preferably 13 to 17. The alkyl moiety may be a straight-chain hydrocarbon or a branched hydrocarbon. The alkyl moiety may be saturated with hydrogen atoms or may contain unsaturations. The stereochemistry around these unsaturations may be in a cis or trans conformation. Examples of alkyl moieties are methyl, ethyl, propyl, butyl, tert-butyl, lauryl (dodecanyl), myristyl (tetradecanyl), palmityl (hexadecanyl), stearyl (octadecanyl), decanyl (eicosanyl), behenyl (docosanyl), and lignoceryl (tetracosanyl).

The term “saturated” as used herein refers to a hydrocarbon moiety having a full compliment of single bonds (alkyl). The term “unsaturated” or “unsaturation” as used herein refers to a hydrocarbon moiety having at least one non-single bond between carbon atoms of the hydrocarbon. An alkyl moiety preferably comprises one unsaturation, although the alkyl moiety can have more than one unsaturation. An unsaturation can exist as a double bond or a triple bond. The unsaturation is preferably a double bond.

The term “cholesterol” as used herein refers to a compound of the following formula:

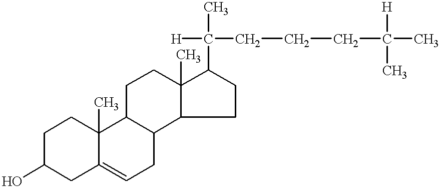

The cholesterol moiety is preferably linked to a compound of the invention through the hydroxyl moiety. The term “cholesterol” as used herein also refers to any derivatives of cholesterol, such as the ester derivative of cholesterol, where the hydroxyl moiety exists as an ester moiety, as well as any additions of chemical substituents to the rings of the cholesterol molecule or its branched hydrocarbon moiety. In addition, the term “cholesterol” refers to other sterols and bile salts, such as cholic acids, deoxycholic acids, ergosterol, glycocholic acid, and taurocholic acid. Other examples of sterols and bile salts can be found in Stryer, Biochemistry, 1988, W. H Freeman and Co., New York.

The term “polyethylene glycol” as used herein refers to multiple types of polymers of varying molecular weight and their derivatives, such as poly(ethylene glycol) monomethyl ether. Other suitable polyethylene glycol (PEG) derivatives include PEG-silicon derived ethers. Many of these polymers are commercially available in a variety of molecular weights. Others may be conveniently prepared from commercially available materials, such as by coupling an amino-PEG moiety to a haloalkyl silyl or silane moiety. When linked to a HGDs of the invention, these PEG moieties improve the solubility of HGDs in aqueous solution. Moreover, the resulting PEG-modified HGDs may exhibit decreased non-specific binding in biological samples or organisms, especially decreased binding to serum albumin.

Suitable PEG molecules may vary in molecular weight from about 200 grams/mole to about 20,000 grams/mole or more. A choice of modifying a HGD of the invention with PEG molecule or molecules of a particular molecular weight may depend upon the degree of hydrophobicity of the HGD.

Amino moieties linked to compounds of the invention, having a formula of —NH₂, are not charged under a neutral pH. However, under acidic conditions, the amino moiety has the formula —N⁺H₃, which bears a positive charge. In addition, secondary and tertiary amine moieties may be uncharged at neutral pH and bear a positive charge under acidic conditions.

Quaternary amine moieties linked to compounds of the invention, bear a positive charge at neutral pH. An example of a quaternary amine is —N⁺(CH₃)₃. Quaternary amine moieties may also exist as a nitrogen linked to at least one alkyl moiety and two or fewer methyl moieties.

A guanidinium moiety having a formula —NH—C(N⁺H₂)—NH₂, can bear a positive charge at neutral pH. The guanidinium moiety can be neutrally charged under alkaline conditions, where the structure is

—NH—C(NH)—NH₂—

The glycosyl moiety of the compound set forth in formula (I) can adopt different conformations. For example, two chair conformational isomers exist for the glycosyl moiety of formula (I), where the chemical substituents exist in either axial or equatorial positions, depending on the isomer. In addition, the glycosyl ring can adopt a boat conformation. Furthermore, the R₁ moiety can exist as either the α or β anomer around the first carbon atom of the ring.

The glycosyl derivative set forth in Formula I can also exist in monomeric form or in an oligomeric form. The glycosyl moieties of oligomers may have the same formula (homogeneous) or have different types of substituents (heterogeneous). The glycosyl moieties of the oligomers may be linked by any of the chemical moieties attached to the ring, including the amine (primary, secondary, tertiary, or quaternary) moiety of the second carbon and any other substituent linked to the one, three, four, and five carbon positions of the glycosyl ring.

In a preferred embodiment, the invention relates to the compound of formula (I), where R₃, R₃′, R₄, R₄′, R₅, and R₅′ are independently selected from the group consisting of hydrogen, —OH, —O—C(O)-alkyl, —O— alkyl, and -alkyl.

In another preferred embodiment, the invention relates to the compound of formula (I), where R₂ and R₂′ are independently selected from the group consisting of hydrogen, —NH₂, —N⁺(CH₃)₃, and —NH—C (N⁺H₂) —NH₂.

In yet another preferred embodiment, the invention relates to the compound of formula (I), where R₁ and R₁′ are independently selected from the group consisting of hydrogen, —OCH₃, -alkyl, —O— alkyl, —O—C(O)-alkyl, —O—CH₂—CH₂ (alkyl) —CH₂ (alkyl), —O—CH₂—CH₂ (O-alkyl) —CH₂ (O-alkyl), —O—CH₂—CH₂ (O—C (O) -alkyl) —CH₂ (O—C (O) -alkyl) —O—(CH₂)ₘ-cholesterol, —O—(CH₂),—NH₂, and —O—(CH₂)ₙ—N⁺(CH₃)₃, where m is selected from the group consisting of 0, 1, 2, 3, 4, and 5, and wherein n is selected from the group consisting of 1, 2, 3, 4, and 5.

The HGDs of the invention can directly target macromolecules to specific cells and even to specific compartments within cells. The HGDs can target macromolecules to specific cells in an unmodified form. Alternatively, any oxygen of the HGD can be modified with molecules that target the HGD to specific cells. These targeting molecules can be tailored to deliver the HGC molecules or compositions to any cell type after systemic delivery. Examples of these cells are endothelial cells, cells involved at the sites of angiogenesis, lymph node cells, and antigen presenting cells. These targeting molecules can be selected from any molecules that bind receptor proteins. Examples of targeting molecules include interleukin-2 (IL-2), interleukin-12 (IL-12), angiostatin, cytosine deaminase, VEGF, α-interferon, β-interferon, γ-interferon, vaccines, antibodies, amino acids, peptides, lytic peptides, binding peptides, peptide hormones, transferrin, lactoferrin, and the Flt-3 ligand. Other examples of targeting molecules are discussed in a U.S. continuation-in-part application with Ser. No. 08/167,641, entitled “Nucleic Acid Transporter Systems and Methods of Use,” invented by Woo et al., filed on Dec. 14, 1993, Attorney Docket No. 205/012, incorporated by reference herein in its entirety including all figures, tables, and drawings.

The term “binding peptide” as used herein refers to a peptide which is capable of binding to a macromolecule. The binding peptide can target the macromolecule to a particular compartment within cells. In addition, binding peptides may bind to macromolecules and condense the macromolecules, such as DNA plasmids. Methods of determining the degree to which a binding peptide condenses macromolecules can be accomplished using dynamic light scattering techniques known by persons of ordinary skill in the art.

The binding peptide can also harbor one or more hydrophobic moieties which enable the peptide to associate with lipid membranes of cells. Binding peptides can include, but are not limited to, components capable of stabilizing and/or condensing nucleic acid molecules by electrostatic binding, hydrophobic binding, hydrogen binding, intercalation or forming helical structures with the macromolecule, preferably a nucleic acid molecule, including interaction with the major and/or minor grove of DNA. The binding peptide can be capable of non-covalently binding to macromolecules, preferably nucleic acid molecules. The binding peptides may also be capable of associating with a surface ligand, a nuclear ligand, and/or a lysis agent.

The term “peptide” as used herein refers to a polymer of amino acids that is preferably less than 40 residues in length, more preferably less than 30 amino acids in length, and most preferably less than 20 amino acids in length.

The term “lytic peptide” as used herein refers to a molecule which is capable of fusing with an endosomal membrane or breaking down an endosomal membrane and freeing the contents into the cytoplasm of the cell. The lytic peptide can contain the following elements: (1) a peptide region capable of lysing endosome complexes; and (2) a hydrophobic region or regions capable of conferring a hydrophobic character to the peptide. The hydrophobic region or regions can enable the peptide to associate with lipid membranes of cells. Hydrophobic moieties of certain lytic peptide are preferably fatty alkyl moieties such as lauryl, myristyl, palmityl, stearyl, arachidyl, behenyl, lignoceryl, palmitolyl, olyl, linolyl, linolyl, and arachidonyl moieties. It is preferred that lytic peptides are pH selective, meaning that the lytic properties of the peptides are inactive at neutral pH, e.g., pH 6 through 9, while active at acidic pH, e.g., pH 6 and below. The lytic peptide may also exist in a constitutively active state. It is also preferred that lytic peptides interact with other components of the invention, such as macromolecules and binding peptides.

In other preferred embodiments, the invention relates to the compound of formula (I), where the alkyl moiety is a straight chain hydrocarbon moiety having 14, 16, or 18 carbon atoms and 0, 1, 2, or 3 unsaturations.

In another preferred embodiment, the invention relates to the compound of formula (II):

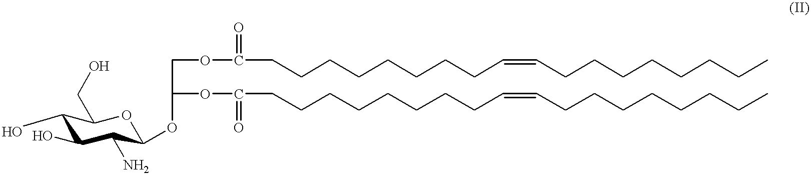

The conformation around the unsaturated portion of each oleyl alkyl moiety may alternately exist in a cis or trans conformation. The conformation noted in Formula II is a cis conformation.

In yet another preferred embodiment, the invention relates to the compound of formula (III):

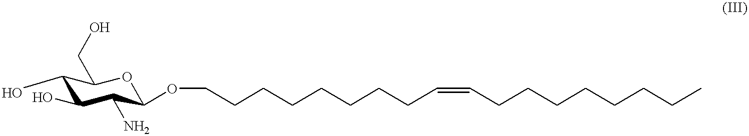

The HGD molecule of formula III can be referred to as 1-mono-oleyl-glucosamine (MOG).

In another preferred embodiment, the invention relates to the compound of formula (IV):

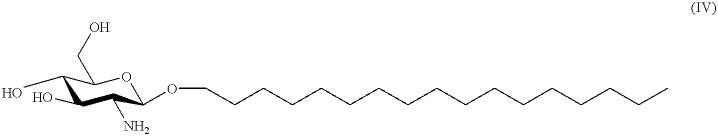

The HGD molecule of formula IV can be referred to as 1-mono-palmityl-glucosamine (MPG).

II. Compositions of the Invention

An added potential benefit of utilizing cationic HGDs lies in the nature of the cationic moiety. Adjusting the pH during formulation of an HGD/DNA complex may control the condensation of the complex (i.e., a highly condensed, a loosely condensed, or a non-condensed complex). For example, MOG formulated with DNA at pH 5 may form a more condensed complex than if it were formulated at pH 7.

Thus in another aspect, the invention features a composition for delivering one or more macromolecules into cells, comprising: (a) a compound comprising a glycosyl moiety having a nitrogen-based substituent linked to a carbon atom within the glycosyl moiety, where the nitrogen-based substituent is selected from the group consisting of

—NH₂, —N⁺(CH₃)₃,

—(CH₂)ₙ—N (R₁₀)₃, and —NH—C(N⁺H₂)—NH₂, and where substituents linked to other carbon atoms within the glycosyl moiety are selected from the group consisting of hydrogen, -alkyl, —O—alkyl, —O—C(O)-alkyl,

—O—CH₂—CH₂ (O—C (O)—R₆) —CH₂ (O—C (O)—R₇), —O—CH₂—CH₂ (OR₆) —CH₂ (OR₇), —O—CH₂—CH₂ (R₆) —CH₂ (R₇), —O—(CH₂)ₘ-cholesterol, polyethylene glycol, —O—(CH₂)ₙ—N (Rs)₃, —NH₂, —N⁺(CH₃)₃, —(CH₂)ₙ—N (R₉)₃ and —(CH₂)—OR₁₀ where R₆, R₇, R₈, R₉, and R₁₀ are independently selected from the group consisting of hydrogen, methyl, and alkyl, and where m is selected from the group consisting of 0, 1, 2, 3, 4, and 5, and where n is selected from the group consisting of 1, 2, 3, 4, and 5; and (b) the macromolecule or macromolecules.

In yet another aspect, the invention features a composition for delivering one or more macromolecules into cells, comprising: (a) a compound having a structure set forth in formula (I), where (i) R₁ and R₁′ are independently selected from the group consisting of hydrogen, —OH, —OCH₃, -alkyl, —O— alkyl, —O—C(O)-alkyl, —O—CH₂—CH₂ (O—C (O)—R₆) —CH₂ (O—C (O)—R₇) , —O—CH₂—CH₂ (OR₆) —CH₂ (OR₇) , —O—CH₂—CH₂ (R₆) —CH₂ (R₇) , —O—(CH₂)ₘ-cholesterol, —O—(CH₂)ₙ—N(R₈)₃, —NH₂, —N⁺(CH₃)₃, (CH₂)ₙ—N (R₉)₃, and —(CH₂)—OR₁₀, where R₆, R₇, R₈, R₉, and R₁₀ are independently selected from the group consisting of hydrogen, methyl, and alkyl, and where m is selected from the group consisting of 0, 1, 2, 3, 4, and 5, and where n is selected from the group consisting of 1, 2, 3, 4, and 5; (ii) R₂ and R₂′ are independently selected from the group consisting of hydrogen, —NH₂, —N⁺(CH₃)₃, —(CH₂)ₙ—N (R₁₀)₃, and —NH—C (N⁺H₂) —NH₂, where R₁₀ is selected from the group consisting of hydrogen, methyl, and alkyl; and (iii) R₃, R₃′, R₄, R₄′, R₅, and R₅′ are independently selected from the group consisting of hydrogen, —OH, —OCH₃, -alkyl, —O— alkyl, —O—C(O)-alkyl, —O—CH₂—CH₂ (O—C (O)—R₆) —CH₂ (O—C (O)—R₇), —O—CH₂—CH₂ (OR₆) —CH₂ (OR₇), —O—CH₂—CH₂ (R₆) —CH₂ (R₇) —O—(CH₂)ₘ-cholesterol, —O—(CH₂)ₙ—N (R₈)₃, —NH₂, —N⁺(CH₃)₃, —(CH₂)ₙ—N (R₉)₃, and —(CH₂) —OR₁₀, where R₆, R₇, R₈, R₉ and R₁₀ are independently selected from the group consisting of hydrogen, methyl, and alkyl, and where m is selected from the group consisting of 0, 1, 2, 3, 4, and 5, and where n is selected from the group consisting of 1, 2, 3, 4, and 5; and (b) the macromolecule or macromolecules.

Any of the HGDs described herein may be utilized in compositions for delivering macromolecules to cells. HGDs that have a R₅′ of —CH₂O—C(O)—(CH₂)ₚCH₃, where p is selected from the group consisting of 10, 12, 14, or 16, a R₃′ moiety that is identical to R₅′, a R₄′ moiety that is —OH, and a R₂′ moiety that is —NH₂, is not utilized for delivery of the enzyme superoxide dismutase. However, any of the other HGDs of the invention can be utilized for the delivery of the enzyme superoxide dismutase or any other molecule to cells.

The term “delivering” as used herein refers to transportation of a molecule to a desired cell or any cell. The macromolecule can be delivered to the cell surface, cell membrane, cell endosome, within the cell membrane, nucleus, or within the nucleus, or any other desired area of the cell. Delivery includes transporting macromolecules such as nucleic acid molecules, proteins, lipids, carbohydrates, and various other molecules. The term “macromolecule” as used herein preferably refers to polymeric compounds. Biological polymers include polypeptides, proteins, lipids, and nucleic acid molecules. The term “macromolecule” as used herein refers to lipids and carbohydrates, preferably peptidomimetics and organic compounds, more preferably proteins and peptides, and most preferably nucleic acid molecules. The term “macromolecule” as used herein also refers to an ionic molecule. Preferably, one type of macromolecule may be transported into cells using the compositions of the invention, although more than one type of macromolecule may be delivered into cells using compositions of the invention.

The term “peptidomimetic” as used herein refers to peptide-like molecules which contain non-hydrolyzable chemical moieties in place of those which exist in naturally occurring peptides. Thus, regions of a peptide which are hydrolyzable such as carboxyl moieties, are replaced by non-hydrolyzable moieties, such as methylene moieties in a peptidomimetic.

The term “anionic molecule” as used herein refers to a molecule that bears at least one negative charge. Examples of anionic molecules are nucleic acid molecules, DNA molecules, RNA molecules, and nucleotide analog molecules.

The term “nucleic acid molecules” as used herein refers to naked DNA, DNA complexed with other molecules, nucleic acid cassettes, naked RNA, RNA complex with other molecules, or nucleic acid molecules contained within vectors, plasmids, and viral nucleic acid molecules.

The term “DNA molecules” as used herein refers to molecules that comprise deoxyribonucleic acids. DNA may be complexed with other molecules such as proteins or other excipients, such as polyvinylpyrrolidone (PVP). The DNA might also be part of polydeoxynucleotides, nucleic acid cassettes, and be utilized as anti-sense DNA molecules. The DNA molecules can exist in single stranded, double stranded, and triple helix forms. These are examples and are not meant to be limiting.

The term “RNA molecules” as used herein refers to ribonucleic acid molecules. The RNA molecules can be complexed with other molecules. The RNA molecules can be ribozymes or be utilized as anti-sense RNA molecules. The RNA molecules can exist in single stranded, double stranded, and triple helix forms. These are examples and are not meant to be limiting.

The term “nucleotide analog molecule” as used herein refers to a chemically-modified form of adenine, thymidine, cytosine, guanine, and uracil. Chemical moieties may be removed or deleted from these nucleotides. Alternatively, a variety of chemical moieties may be added to these nucleotides. The nucleotide analog molecules may exist in mononucleotide form or as polymers comprising one or more nucleotide analogs among other nucleotide molecules.

In a preferred embodiment, the invention relates to the composition, where R₃, R₃′, R₄, R₄′, R₅, and R₅′ are independently selected from the group consisting of hydrogen, —OH, —O—C(O)-alkyl, —O— alkyl, and -alkyl.

In another preferred embodiment, the invention relates to the composition, where R₂ and R₂′ are independently selected from the group consisting of hydrogen, —NH₂, —N⁺(CH₃)₃, and —NH—C(N⁺H₂) —NH₂.

In yet another preferred embodiment, the invention relates to the composition, where R₁ and R₁′ are independently selected from the group consisting of —OCH₃, -alkyl, —O— alkyl, —O—C(O)-alkyl, —O—CH₂—CH₂(alkyl)—CH₂ (alkyl) ,—O—CH₂—CH₂ (O-alkyl) —CH₂ (O-alkyl), —O—CH₂—CH₂ (O—C (O)-alkyl) —CH₂ (O—C (O)-alkyl), —O—(CH₂)ₘ-cholesterol, —O—(CH₂)ₙ—NH₂, and —O—(CH₂)ₙ—N⁺(CH₃)₃, where m is selected from the group consisting of 0, 1, 2, 3, 4, and 5, and where n is selected from the group consisting of 1, 2, 3, 4, and 5; In other preferred embodiments, the invention relates to the composition, where the alkyl moiety is a straight chain hydrocarbon moiety having 14, 16, or 18 carbon atoms and 0, 1, 2, or 3 unsaturations.

In another preferred embodiment, the invention relates to the composition, where the compound has a structure set forth in formula (II).

In another preferred embodiment, the invention relates to the composition, where the compound has a structure set forth in formula (III)

In a preferred embodiment, the invention relates to the composition, where the compound has a structure set forth in formula (IV).

In yet another preferred embodiment, the invention relates to the composition, where the macromolecule is an anionic molecule.

In another preferred embodiment, the invention relates to the composition, where the anionic molecule is selected from the group consisting of a polynucleic acid molecule, a DNA molecule, a RNA molecule, and a nucleotide analog molecule.

In yet another preferred embodiment, the invention relates to the composition, where the DNA molecule is a plasmid molecule comprising at least one element for polypeptide expression in eukaryotic cells.

The term “plasmid molecule” as used herein refers to a construct comprising genetic material (i.e., nucleic acids). A plasmid molecule includes genetic elements arranged such that an inserted coding sequence can be transcribed in eukaryotic cells. In addition, at least a portion of the plasmid may be able to incorporate in random or defined regions within cellular genomic DNA. Alternatively, the plasmid may replicate autonomously of the cellular genomic DNA. Also, while the plasmid may include a sequence from a viral nucleic acid molecule, such a viral sequence may not cause the incorporation of the plasmid into a viral particle, and the plasmid is therefore a non-viral vector. Preferably, a plasmid is a closed circular DNA molecule.

The term “element for polypeptide expression” as used herein refers to a portion on the plasmid made up of nucleic acid molecules, that promotes the transcription of a nucleotide sequence of the plasmid into RNA. If the RNA is message RNA, the message RNA is typically translated into a polypeptide. In some cases, an RNA product may have relevant activity (i.e., a ribozyme) and could thus be regarded as a gene product. The process of expression may involve further processing steps to the RNA product of transcription, such as splicing to remove introns, and/or post-translational processing of a polypeptide product. The term “element for polypeptide expression” may also be referred to as an expression cassette. An expression cassette contains at least one coding sequence along with sequence elements which direct the initiation and termination of transcription. A transcription unit may however include additional sequences, which may include sequences involved in post-transcriptional or post-translational processes.

The term “coding region” or “coding sequence” refers to a nucleic acid sequence which encodes a particular gene product for which expression is desired, according to the normal base pairing and codon usage relationships. Thus, the coding sequence must be placed in such a relationship to transcriptional control elements and to translational initiation and termination codons such that a proper length transcript will be produced and will result in translation in the appropriate reading frame to produce a functionally desired product.

In other preferred embodiments, the invention relates to the composition, where the plasmid molecule further comprises a gene encoding IL-2.

The term “IL-2” as used herein refers to interleukin-2. Interleukin-2 is a T-cell growth factor. The function of IL-2, among others, involves stimulating the proliferation of helper T-cells, in particular T_{H}1 cells by an auto-immune mechanism. Secretion of IL-2 will also stimulate the proliferation of other activated helper T-cells and cytotoxic T cells. An amino acid sequence of IL-2 is described herein.

In another preferred embodiment, the invention relates to the composition, further comprising at least one co-lipid.

The term “co-lipid” as used herein refers to a hydrophobic molecule which can be formulated into compositions in conjunction with HGDs of the invention. Examples of co-lipids are phospholipids, fatty acids, cholesterol, and cholesterol derivatives.

In yet another preferred embodiment, the invention relates to the composition, where the co-lipid is DOPE.

The term “DOPE” as used herein refers to dioleoylphosphatidyl ethanolamine. This phospholipid is zwitterionic, indicating that it can harbor both a positive and a negative charge at neutral pH. Examples provided herein demonstrate that compositions comprising HGDs of the invention and DOPE molecules may form liposomes of appropriate diameters for the delivery of the DNA plasmids into cells. As mentioned above, however, the HGDs of the invention can also adopt micelle conformations, and can form condensed and non-condensed DNA/HGD complexes.

In other preferred embodiments, the invention relates to the composition, where the co-lipid is cholesterol.

In another preferred embodiment, the invention relates to the composition, further comprising a cryoprotectant.

The term “cryoprotectant” as used herein refers to a chemical or compound which can protect nucleic acid molecules and composition of the invention during formulation, storage, and rehydration processes. Examples of cryoprotectants include, but are not limited to, such compounds as lactose, sucrose, mannitol, trehalose, and polyvinylpyrrolidone. Polyvinylpyrrolidone is referred to as PVP herein.

In yet another preferred embodiment, the invention relates to the composition, where the cryoprotectant is PVP.

In other preferred embodiments, the invention relates to the composition, where the composition is capable of forming liposomes. As mentioned above, the HGDs can adopt micelle conformations. The propensity with which a HGD formulation adopts a micellular structure or a liposomal structure can depend upon the size and structure of the side chains emanating from the glycosylamine base as well as the presence and concentration of co-lipid. When a macromolecule or macromolecules are added to the HGDs of the invention, condensed and non-condensed DNA/HGD complexes can be formed for the effective delivery of macromolecules into cells, including cells within tumors.

The term “liposome” as used herein refers to a complex which is characterized as a sphere-like molecule. The sphere-like molecule can harbor a high concentration of lipid moieties on the surface of the molecule and a high concentration of macromolecules on the interior of the sphere. Liposomes may also deliver macromolecules into cells by surrounding the surface of the macromolecule. Liposomes can be formed by HGDs of the invention as well as complexes with co-lipid molecules of the invention.

The term “condensation” as used herein refers to charge neutralization, exclusion of water, and compacting a macromolecule or macromolecules into a smaller volume than they occupied prior to complexation with an HGD of the invention. The HGDs that form condensed and non-condensed complexes with macromolecules may also achieve one or more of the following effects, due to their physical, chemical, or rheological properties:

1. protect nucleic acid molecules; such as plasmid DNA, from nuclease activity;

2. increase the area of contact between nucleic acid molecules, such as DNA plasmids, through extra-cellular matrices and cellular membranes, into which the nucleic acid molecules may be taken up by the cells;

3. concentrate nucleic acid molecules, such as DNA plasmids, at the cell surfaces due to water exclusion; and

4. indirectly facilitate uptake of nucleic acid molecules, such as DNA plasmids, by either increasing interactions with cellular membranes and/or by perturbing cellular membranes due to osmotic, hydrophobic, or ionic effects.

The degree of condensation can have an effect upon the efficiency with which compositions of the invention deliver macromolecules into cells. For example, condensed complexes can enhance macromolecule delivery into cells for in vitro delivery. However, complexes exhibiting a lower degree of condensation can enhance macromolecule delivery into cells for in vivo delivery, especially in tumors.

In another preferred embodiment, the invention relates to the composition, where the composition has an effective diameter between 100 nanometers and 300 nanometers. The effective diameter of complexes can be measured by techniques well-known to persons of ordinary skill in the art, such as by using dynamic light-scattering techniques. These complexes may be characterized as having partial liposomal structure and/or partial micellular structure.

The term “effective diameter” refers to the spherical volume that a composition of the invention occupies. A rod-shaped composition of the invention occupies a spherical volume in solution due to the tumbling of the composition in a solvent environment. The length of a rod-shaped composition can define the effective diameter of the spherical volume occupied by the composition.

In yet another preferred embodiment, the invention relates to the composition, where the composition has a charge ratio selected from the group consisting of 1:0.5, 1:1, 1:2, 1:3, 1:4, 1:5, 1:6, and 1:9.

The term “−/+ charge ratio” as used herein refers to the ratio between the net negative charges on the macromolecules to the net positive charges on molecules complexing the macromolecules. The −/+ charge ratio can be changed depending upon the ratios of different molecules mixed in the composition.

III. Methods of Delivering Macromolecules

In another aspect, the invention features a method for delivering macromolecules to one or more cells of a mammal. The method comprises the step of administering the composition of the invention to the cells.

The term “administering” as used herein refers to a procedure for introducing a macromolecule, preferably a nucleic acid molecule, into the body of an organism. The composition can be administered directly to a target tissue or administered by systemic delivery. Specifically, administration may be accomplished by direct injection into tissue. A molecular complex may also be administered intravenously, intramuscularly, by hypospray, or in conjunction with various excipients, such as polyvinylpyrrolidone (PVP). Routes of administration include intramuscular, intramural, aerosol, oral, topical, systemic, nasal, ocular, parenteral, intraperitoneal, and/or intratracheal. Compositions of the invention can be administered to an organism by direct injection into the organism or by removing cells from an organism and transforming these cells with the nucleic acid molecules delivered by the methods and compositions of the invention.

As used herein, “transformation” or “transformed” is a mechanism of gene transfer which involves the uptake of nucleic acid molecules by a cell or organism. It is a process or mechanism of inducing transient or permanent changes in the characteristics (expressed phenotype) of a cell. Such changes are by a mechanism of gene transfer whereby nucleic acid molecules are introduced into a cell in a form where they can express a specific gene product or alter the expression of, or effect, of endogenous gene products.

The term “mammal” as used herein refers to any warm blooded organism. Such organisms are preferably mice, rats, rabbits, guinea pigs, and goats, more preferably cats, dogs, monkeys and apes, and most preferably humans.

In a preferred embodiment, the invention relates to a method for delivering macromolecules to cells, where the composition is administered to the cells in vitro.

The term “cells in vitro” as used herein refers to cultured cells. The cells may have been removed from an organism and placed in a culture dish with an appropriate medium that maintains the cellular integrity. These types of cells may be part of a tissue or may exist as rapidly dividing cells that form monolayers in culture dishes. These cells may optionally be placed into an organism after administration of the composition.

In another preferred embodiment, the invention relates to a method for delivering macromolecules to cells, where the composition is administered to the cells in vivo.

The term “cells in vivo” as used herein refers to cells which exist inside of an organism. The cells may be of the same or different origin as the organism. For example, cultured cells from one organism may be placed and grown within another organism.

In yet another preferred embodiment, the invention relates to a method for delivering macromolecules to cells, where the administration results in IL-2 expression in the cells.

In other preferred embodiments, the invention relates to a method for delivering macromolecules to cells, where the composition is administered by the techniques set forth by the group consisting of direct injection to a tissue, parenteral injection, intravenous injection, oral administration, and administration by inhalation.

IV. Methods for Synthesizing Compounds of the Invention

In yet another aspect, the invention features a method for synthesizing a compound of the invention, comprising the steps of: (a) reacting a first reactant of formula (V):

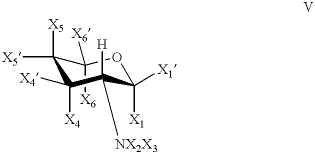

with a second reactant, where X₁ and X₁′ are independently selected from the group consisting of hydrogen, halogen atom, and an activatable moiety; X₂ and X₃ are independently selected from the group consisting of a protecting moiety, hydrogen, halogen, or any activatable moiety; and where X₄, X₄′, X₅, X₅′, X₆ and X₆′ are independently selected from the group consisting of hydrogen, —O—acetyl, —OH, —CH₂—O—acetyl, —CH₂—OH, and —O—alkyl; where the second reactant is selected from the group consisting of HOCH₃, HO—alkyl, HO—C(O)-alkyl, HO—CH₂—CH₂ (O—C (O) -R₆) —CH₂ (O—C (O)—R₇), HO—CH₂—CH₂ (OR₆) —CH₂ (OR₇), HO—CH₂—CH₂ (R₆) —CH₂ (R₇), HO—(CH₂)ₘ-cholesterol, and HO—(CH₂)ₙ—N(R₈)₃, where R₆, R₇, R₈, and R₉are independently selected from the group consisting of hydrogen, methyl, and alkyl, and where m is selected from the group consisting of 0, 1, 2, 3, 4, and 5, and where n is selected from the group consisting of 1, 2, 3, 4, and 5; (b) reacting the product of step (a) with a reducing agent; and (c) purifying the compound of the invention.

The term “activatable moiety” as used herein refers to alkyl, —N═N, aryl-thio, O-acyl, trichloroimidate, O-pentyl, O-sulfonyl, O-silyl, and any of the activatable moieties set forth in Toshima and Tatsuta, 1993, “Recent Progress in O-Glycosylation Methods and Its Application to Natural Product Synthesis,” Chem. Rev. 93: 1503-1531 incorporated by reference in its entirety, including all figures, tables, and drawings.

The term “protecting group” as used herein refers to a chemical moiety that can be linked to the HGD in set of reaction conditions and then be removed from the HGD in another set of reaction conditions. Many types of protecting groups are known to persons of ordinary skill in the art. Examples of protecting groups are carbamates, such as t-butyl, benzyl, and 9-fluorenylmethyl; amides, such as N-trifluoroacetyl and N-acetyl; cyclic imides, such as N-phthalimide and N-dithiasuccinimide; esters, such as formate, acetate, and benzoate; carbonates, such as methyl and 9-fluorenylmethyl; and cyclic acetals and ketals, such as methylene, benzylidene, and methoxymethylene. Other examples of protecting groups are described in Green and Wuts, 1991, “Protective Groups in Organic Synthesis,” 2nd Ed., John Wiley & Sons, incorporated by reference herein in its entirety, including all figures, tables, and drawings.

The term “catalyst” as used herein refers to a chemical molecule, that when added to a group of reactants, can increase the rate at which the reactants convert into products. Many types of catalysts are well known to persons of ordinary skill in the art. Examples of catalysts are AgSO₂CF₃, AgClO₄, HgCl₂, HgBr₂, SnCl₄, FeCl₃, BF₃—OEt₂, TMS—OTf, TsOH, NIS—TfOH. Other examples of catalysts are discussed in the Toshima and Tatsuta publication, previously incorporated by reference herein.

In another aspect, the invention features a method for synthesizing a compound of claim 1, comprising the steps of: (a) reacting a first reactant of formula (V) with a second reactant, where X₁ and X₁′ are independently selected from the group consisting of hydrogen, —OCH₃, -alkyl, —O— alkyl, —O—C(O)-alkyl, —O—CH₂—CH₂ (O—C (O) —R₆) —CH₂ (O—C (O) —R₇) , —O—CH₂—CH₂ (OR₆) —CH₂ (OR₇) , —O—CH₂—CH₂ (R₆) —CH₂ (R₇) , —O—(CH₂)ₘ-cholesterol, —O—(CH₂)ₙ—N(R₈)₃, —NH₂, —N⁺(CH₃)₃, and —(CH₂)ₙ—N(R₉)₃, where R₆, R₇, R₈, and R₉are independently selected from the group consisting of hydrogen, methyl, and alkyl, and where m is selected from the group consisting of 0, 1, 2, 3, 4, and 5, and where n is selected from the group consisting of 1, 2, 3, 4, and 5; and where X₂ and X₃ are independently selected from the group consisting of hydrogen and a protecting group, and X₄, X₄¹′ X₅, X₅, X₅′, X₆, and X₆′ are independently selected from the group consisting of hydrogen, —OH, and —O— alkyl; where the second reactant is selected from the group consisting of ClCH₃,

Cl-alkyl,

Cl—CH₂—CH₂ (O—C (O)—R₆) —CH₂ (O—C (O) —R₇) ,

Cl—CH₂—CH₂ (OR₆) —CH₂ (OR₇) , Cl—CH₂—CH₂ (R₆) —CH₂ (R₇) , Cl-(CH₂)ₘ-cholesterol, and Cl-(CH₂)ₙ—N(R₈)₃, where R_{6,} R₇, R₈, and R₉are independently selected from the group consisting of hydrogen, methyl, and alkyl, and where m is selected from the group consisting of 0, 1, 2, 3, 4, and 5, and where n is selected from the group consisting of 1, 2, 3, 4, and 5; (b) reacting the product of step (a) with a reducing agent and a catalyst; and (c) purifying the compound of the invention.

In a preferred embodiment, the invention relates to the methods for synthesizing compounds of the invention, where the protecting group is an N-phthalimido moiety. A N-pthalimido moiety has the following general structure:

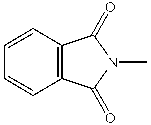

In a preferred embodiment, the invention relates to the methods for synthesizing compounds of the invention, where the reducing agent is selected from the group consisting of H₂NNH₂, H₂, and NABH₄.

In a preferred embodiment, the invention relates to the methods for synthesizing compounds of the invention, where the catalyst is palladium.

The summary of the invention described above is non-limiting and other features and advantages of the invention will be apparent from the following detailed description of the preferred embodiments, as well as from the claims.

### DETAILED DESCRIPTION OF THE PREFERRED EMBODIMENTS

I. Theory And Operation Of Invention

The following explanation of the invention is to aid in understanding various aspects of the invention. The following explanation does not limit the operation of the invention to any one theory.

An important goal of the current invention is to increase the efficacy of gene delivery and gene expression in target cells. Gene delivery is the first step in the process of ultimately obtaining expression of a product encoded by a nucleic acid molecule targeted for delivery to a cell. One method of improving gene delivery is to enhance the uptake of nucleic acid molecules by cells. Uptake of nucleic acid molecules by cells is dependent upon a number of factors, one of which is the size of the composition carrying the nucleic acid molecule or oligonucleotide to be expressed in the target cell. For instance, some investigators report a positive correlation between the degree of condensation of DNA in a complex to be delivered to a cell and the efficiency of macromolecule uptake. Wagner et al., 1991, Proc. Natl. Acad. Sci. Vol 88. Accordingly, it would be desirable to find a substance able to complex and condense nucleic acid molecules, protect them from degradation by nucleases, and enhance uptake of the nucleic acid molecules by the target cells by either non-specific adsorptive mechanisms or receptor mediated endocytosis. In addition, adding other moieties to the composition can enhance the ability of the composition to obtain expression of the product targeted to cells. Furthermore, these substances should be readily available, biocompatible and capable of being modified to alter their physical, chemical, and physiological properties. Such substances should be able to form compositions suitable for administration to an organism by various means such as, but not limited to, injection or oral delivery while maintaining or regaining the physical characteristics necessary to increase cellular uptake and expression of nucleic acids or oligonucleotides.

HGDs of the invention are such substances. The embodiments and examples below demonstrate how specific HGD compositions stabilize and condense nucleic acid molecules for cell delivery. Furthermore, these embodiments and examples demonstrate how surface and nuclear ligands can be used to target nucleic acid molecules into cellular interior and/or the cell nuclei. Such targeted delivery can be enhanced by use of a lipophilic lytic peptides and lipophilic condensing peptides. It was found that though in vitro transfection results do not necessarily predict effective in vivo delivery, the HGDs can be used in compositions which enhance in vivo delivery, as well as in vitro transfection of nucleic acid molecules. Thus, the embodiments and examples include in vivo and in vitro techniques, various cellular or animal models and methods for inserting nucleic acid into cells.

Also supplied below are embodiments and examples of specific HGD compositions that can be used to provide certain functionalities to the associated nucleic acid in the composition, and thus within a transformed cell or animal containing such a cell. Those in the art will recognize that specific moieties of the HGDs can be identified as having the functional regions providing the desirable properties of the composition. Such regions can be readily minimized using routine deletion, mutation, or modification techniques or their equivalent.

II. Macromolecules of the Invention

A variety of macromolecules can be delivered to cells using the molecular complexes of the invention. These macromolecules include proteins, peptides, lipids, carbohydrates, peptidomimetics, organic molecules, and preferably nucleic acid molecules. A preferred type of polypeptide delivered to cells using the molecular complexes of the invention are toxins, such as ricin and other cytotoxic agents. The specific delivery of toxins to cells can potentially eradicate harmful cells in an organism. This application is particularly useful in the treatment of certain cancers.

The term “nucleic acid molecules” as used herein can refer to DNA and RNA molecules. The nucleic acid molecules can exist in a state in which they are complexed to other molecules, such as radio-labels or dyes. These molecules can be associated with the nucleic acid molecules in a covalent or non-covalent reversibly associated fashion.

The nucleic acid molecules may exist as recombinant vectors containing a variety of nucleic acid elements. These elements can include promoter elements, ribosome binding elements, drug resistance elements, replication binding elements, and genes.

A variety of proteins and polypeptides can be encoded by a gene harbored within a nucleic acid molecule of the invention. Those proteins or polypeptides include hormones, growth factors, enzymes, clotting factors, apolipoproteins, receptors, drugs, oncogenes, tumor antigens, tumor suppressors, cytokines, viral antigens, parasitic antigens, bacterial antigens and chemically synthesized polymers and polymers biosynthesized and/or modified by chemical, cellular and/or enzymatic processes. Specific examples of these compounds include proinsulin, insulin, growth hormone, androgen receptors, insulin-like growth factor I, insulin-like growth factor II, insulin growth factor binding proteins, epidermal growth factor, TGF-α, TGF-β, dermal growth factor (PDGF), angiogenesis factors (acidic fibroblast growth factor, basic fibroblast growth factor and angiogenin), matrix proteins (Type IV collagen, Type VII collagen, laminin), oncogenes (ras, fos, myc, erb, src, sis, jun), E6 or E7 transforming sequence, p53 protein, cytokine receptor, IL-1, IL-6, IL-8, IL-2, α, β, or γIFN, GMCSF, GCSF, viral capsid protein, and proteins from viral, bacterial and parasitic organisms. Other specific proteins or polypeptides which can be expressed include: phenylalanine hydroxylase, α-1-antitrypsin, cholesterol-7α-hydroxylase, truncated apolipoprotein B, lipoprotein lipase, apolipoprotein E, apolipoprotein Al, LDL receptor, scavenger receptor for oxidized lipoproteins, molecular variants of each, VEGF, and combinations thereof. Other examples are clotting factors, apolipoproteins, drugs, tumor antigens, viral antigens, parasitic antigens, and bacterial antigens. Other examples can be found above in the discussion of nucleic acid. One skilled in the art readily appreciates that these proteins belong to a wide variety of classes of proteins, and that other proteins within these classes can also be used. These are only examples and are not meant to be limiting in any way.

It should also be noted that the genetic material which is incorporated into the cells from the molecular complex includes (1) nucleic acid molecules not normally found in the cells; (2) nucleic acid molecules which are normally found in the cells but not expressed at physiological significant levels; (3) nucleic acid molecules normally found in the cells and normally expressed at physiological desired levels; (4) other nucleic acid molecules which can be modified for expression in cells; and (5) any combination of the above.

In addition, the macromolecules of the invention may relate to nucleic acid molecules that can cleave RNA molecules in specific regions. The nucleic acid molecules which can cleave RNA molecules are referred to in art as ribozymes, which are RNA molecules themselves. Ribozymes can bind to discrete regions on a RNA molecule, and then specifically cleave a region within that binding region or adjacent to the binding region. Ribozyme techniques can thereby decrease the amount of polypeptide translated from formerly intact message RNA molecules. The methods of the invention can enhance the delivery of ribozyme nucleic acid molecules to cells using the compositions described herein.

Furthermore, the macromolecules of the invention may relate to nucleic acid molecules which can bind to specific RNA sequences or DNA sequences. Nucleic acid molecules which are designed to specifically bind to regions on RNA molecules or DNA molecules are utilized in antisense techniques. Antisense techniques include the delivery of RNA or DNA to cells that are homologous to message RNA sequences in the cell or two specific sequences in genomic DNA. The antisense nucleic acid molecules bind to the message RNA molecules and block the translation of these message RNA molecules. Antisense techniques can thereby block or partially block the synthesis of particular polypeptides in cells. The methods of the invention can enhance the delivery of antisense nucleic acid molecules to cells using the compositions described herein.

III. Hydrophobic Glycosylamine Derivatives of the Invention

A number of possible HGDs of the invention are illustrated in the following diagram. R represents a lipophilic modification such as, but not limited to C₉-C₂₄ alkyl or acyl chains with or without varying degrees of unsaturation. The amine moiety as shown, may or may not be alkylated. Alkylation of an amine can confer a positive charge on the glycosylamine molecules. As an example, where trimethylamino functionalities are shown, any appropriate alkyl group may be introduced to give di-, tri- or tetra-modified amines. Other examples of lipophilic species are also shown, for example cholesterol or some other hydrophobic group may be introduced directly onto the sugar or attached to a suitable spacer. In addition, the glycosylamine molecules of the invention may be modified with multiple polyethylene glycol moieties or derivatives of polyethylene molecules.

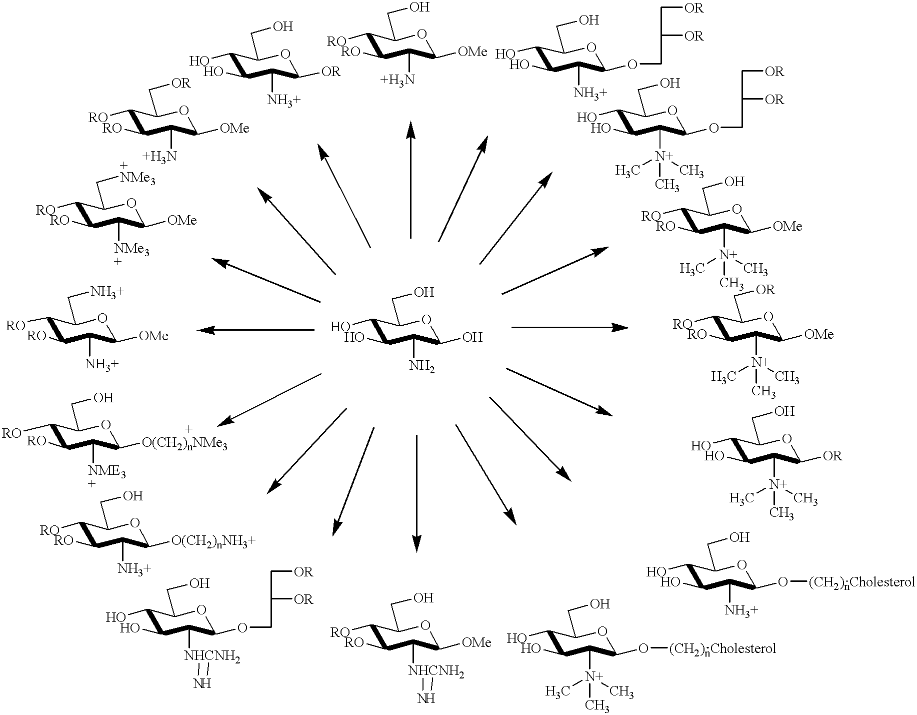

IV. Utility Of The Invention

The compositions of the present invention enhance delivery of macromolecules into cells. The enhanced delivery can be a function of delivering condensed nucleic acid molecules into the nucleus of cells. These compositions can be used to treat diseases by enhancing the delivery of specific nucleic acid molecules to appropriately targeted cells. These compositions can also be used to create transformed cells, as well as transgenic animals for assessing human disease in an animal model.

The present invention features the use of compositions of HGDs with nucleic acid molecules noncovalently bound to the HGDs. The glycosylamine derivatives are capable of condensing the nucleic acid molecules or oligonucleotides. These HGDs provide small, condensed compositions, or reduced diameter compositions, and more stable nucleic acid particles for delivery, thereby enhancing the transfection efficiency of nucleic acid molecules into cells and their nuclei.

By taking advantage of the characteristics of hydrophobic glycosylamine compositions, the present invention enhances delivery of nucleic acid molecules to cells. The components of the compositions can be used alone, together or with other components of a nucleic acid carrier as disclosed in PCT publication WO 93/18759, Woo et al., entitled “A DNA Carrier System and Method of Use,” the whole of which (including drawings) is hereby incorporated by reference in its entirety. The HGD compositions may also comprise lipophilic condensing peptides and/or lipophilic lytic peptides, which can enhance the delivery of nucleic acid molecules to cells by facilitating the release of stable, condensed nucleic acid molecules from endosomes into the interior of cells. These molecules are described in U.S. patent application Ser. No. 08/584,043, titled “Lipophilic Peptides for Macromolecule Delivery” filed on Jan. 11, 1995, incorporated by reference herein in its entirety, including all figures and drawings. This application also discloses examples of receptor ligands and nuclear ligands, which may also comprise the glycosylamine derivative compositions described herein. These targeting ligands are capable of binding to a cell surface receptor and entering a cell through cytotic mechanisms (e.g., endocytosis, potocytosis, pinocytosis). By using targeting ligands specific to certain cells, nucleic acid molecules can be delivered directly to the desired tissue using the hydrophobic glycosylamine compositions. In addition, nuclear localization signals are capable of recognizing and transporting nucleic acid molecules through the nuclear membranes to the nucleus of the cells.

The advantages provided by the above-described hydrophobic glycosylamine compositions can dissect molecular carcinogenesis and disease, assess potential chemical and physical carcinogens and tumor promoters, explore model therapeutic avenues for humans, household pets, as well as livestock and agricultural purposes by applying the components and techniques described herein to transgenic animal models. Furthermore, the above-described hydrophobic glycosylamine compositions permit methods for administration and treatment of various diseases. In addition, the above HGD compositions can transform cells to produce particular proteins, polypeptides, and/or RNA. Likewise, HGD compositions can be used in vitro with tissue culture cells. In vitro uses allow the role of various nucleic acids to be studied by targeting specific expression of RNA or polypetides to specifically targeted tissue culture cells.

The present invention also encompasses transgenic animals whose cells contain the nucleic acid molecules referenced above, which may be delivered via the HGD compositions of the invention. These cells include germ-line or somatic cells. Transgenic animal models can be used for dissection of molecular carcinogenesis and disease, assessing potential chemical and physical carcinogens and tumor promoters, exploring model therapeutic avenues and livestock agricultural purposes.

The methods of use also include a method of treating or preventing human disease, which is another aspect of the present invention. The method of treatment or prevention includes the steps of administering the HGD compositions as described herein to deliver macromolecules to a cell or tissue. Method of treatment may include the transcription or translation of products from the nucleic acid molecules delivered to the cells or tissues. Cell or tissue types of interest can include, but are not limited to: liver, muscle, lung, endothelium, joints, skin, bone, tumors, and blood.

The term “preventing” as used herein refers to the method of the invention decreasing the probability that an organism contracts or develops the abnormal condition.

The term “treating” as used herein refers to the method of the invention having a therapeutic effect and at least partially alleviating or abrogating the abnormal condition in the organism.

The term “therapeutic effect” as used herein refers to relieving to some extent one or more of the symptoms of the abnormal condition; With respect to cancer, the term “therapeutic effect” can refer to the inhibition of cell growth causing or contributing to the cancerous abnormal condition. The term “therapeutic effect” can also refer to the inhibition of growth factors causing or contributing to the abnormal condition. In reference to the treatment of a cancer, a therapeutic effect refers to one or more of the following: (a) a reduction in tumor size; (b) inhibition (i.e., slowing or stopping) tumor metastasis; (c) inhibition of tumor growth; and (d) relieving to some extent one or more of the symptoms associated with the abnormal condition. Compounds demonstrating efficacy against leukemias can be identified as described herein, except that rather than inhibiting metastasis, the compounds may instead slow or decrease cell proliferation or cell growth.

The methods of treatment or use include methods for delivering nucleic acid molecules into a hepatocyte by contacting a hepatocyte with the above-referenced HGD compositions. A surface ligand appropriate for this application is one specific for recognition by hepatocyte receptors. In particular, the asialoorosomucoid protein or galactose may be utilized as a cell surface ligand. Furthermore, these methods of use also include delivery of nucleic acid molecules using a hydrophobic glycosylamine composition of the invention, which may further comprise the lipophilic lytic or lipophilic condensing peptides or receptor or nuclear ligands described herein.

An embodiment of the methods of treatment or use includes a method for delivering macromolecules to muscle cells by contacting the muscle cell with one of the above referenced hydrophobic glycosylamine compositions. The surface ligand used is specific for receptors contained in the muscle cell. In particular, the surface ligand can be insulin-like growth factor-I. Furthermore, the methods of treatment or use may also include delivery of nucleic acid molecules by using a HGD-based composition. The term “muscle cell” as used herein refers to cells associated with skeletal muscle, smooth muscle or cardiac muscle.

Another embodiment of the methods of treatment or use includes a method for delivering macromolecules to bone-forming cells by contacting the bone-forming cells with the hydrophobic glycosylamine compositions of the invention. A surface ligand that can be utilized in conjunction with hydrophobic glycosylamine compositions is specific for receptors associated with bone-forming cells. In particular, surface ligands can include, but are not limited to, bone morphogenetic protein or cartilage induction factor. In addition, glycosylamine derivative compositions may also include lipophilic peptides and/or lytic peptides as described above. As used herein, the term “bone-forming cell” refers to those cells which promote bone growth. Non-limiting examples include osteoblasts, stromal cells, inducible osteoprogenitor cells, determined osteoprogenitor cells, chondrocytes, as well as other cells capable of aiding bone formation.

Still another related embodiment of the methods of treatment or use includes a method for delivering nucleic acid molecules to synoviocytes or macrophages using the above referenced HGD compositions. The HGD composition can comprise lipophilic condensing peptides or lipophilic lytic peptides as described above. The term “synoviocytes” refers to cells associated with the joints or with the fluid space of the joints.

In addition to the above methods, the method of use also includes delivery of macromolecules to cells by administering hydrophobic glycosylamine compositions comprising nuclear ligand binding complexes. Such nuclear carriers would help direct the hydrophobic glycosylamine composition to the nuclei of cells. Furthermore, the above methods of use also include hydrophobic glycosylamine compositions that may also comprise any of the following molecules: lipophilic lytic peptides, lipophilic condensing peptides, receptor ligands, and nuclear ligands.

V. IL-2 Coding Sequences

A preferred embodiment of the invention includes HGD compositions which comprise a DNA plasmid harboring an expression cassette with a gene encoding interleukin-2 (IL-2).

The nucleotide sequence of a natural human IL-2 coding sequences is known, and is provided below, along with a synthetic sequence which also codes for human IL-2.

In some cases, instead of the natural sequence coding for IL-2, it is advantageous to utilize synthetic sequences which encode IL-2. Such synthetic sequences may incorporate alternate codon usage from the natural sequence, and thus have dramatically different nucleotide sequences from the natural sequence. In particular, synthetic sequences can be used which have codon usage at least partially optimized for expression of the IL-2 polypeptide in a human. The natural sequences do not have such optimal codon usage. Preferably, substantially all the codons are optimized.

Optimal codon usage in humans is indicated by codon usage frequencies for highly expressed human genes. The codons, which are most frequently used in highly expressed human genes, are presumptively the optimal codons for expression in human host cells, and thus form the basis for constructing a synthetic coding sequence. An example of a synthetic IL-2 coding sequence is shown as the bottom sequence in Table I.

However, rather than a sequence having fully optimized codon usage, it may be desirable to utilize an IL-2 encoding sequence which has optimized codon usage except in areas where the same amino acid is too close together or abundant to make uniform codon usage optimal.

In addition, other synthetic sequences can be used which have substantial portions of the codon usage optimized, for example, with at least 50%, 70%, 80% or 90% optimized codons as compared to a natural coding sequence. Other particular synthetic sequences for IL-2 can be selected by reference to a codon usage chart. A sequence is selected by choosing a codon for each of the amino acids of the polypeptide sequences. DNA molecules corresponding to each of the polypeptides can then be constructed by routine chemical synthesis methods. For example, shorter oligonucleotides can be synthesized, and then ligated in the appropriate relationships to construct the full-length coding sequences.

TABLE I
HOMOLOGY OF WILD TYPE AND OPTIMIZED IL-2

| | | | |
|---|---|---|---|
| Gap Weight: | 5.000 | Average Match: | 1.000 |
| Length Weight: | 0.300 | Average Mismatch: | −0.900 |
| Quality: | 235.9 | Length: | 462 |
| Ratio: | 0.511 | Gaps: | 0 |
| Percent Similarity: | 74.242 | Percent Identity: | 74.242 |

| | | |
|---|---|---|
| TOP: | WILD TYPE | SEQ ID NO. 1 |
| BOTTOM: | OPTIMIZED | SEQ ID NO. 2 |

| | | |
|---|---|---|
| | . . . . . | |
| 639 | ATGTACAGGATGCAACTCCTGTCTTGCATTGCACTAAGTCTTGCACTTGT | 688 |
| | \|\|\|\|\|\| \| \|\|\|\|\| \|\| \|\|\| \|\|\|\|\| \|\| \|\| \|\| \|\| \|\| \|\| \|\| | |
| 1 | ATGTACCGCATGCAGCTGCTGAGCTGCATCGCCCTGAGCCTGGCCCTGGT | 50 |
| | . . . . . | |
| 689 | CACAAACAGTGCACCTACTTCAAGTTCTACAAAGAAAACACAGCTACAAC | 738 |
| | \|\| \|\|\|\|\| \|\| \|\| \|\| \|\| \|\| \|\|\|\|\| \|\| \|\|\|\|\| \|\| \| | |
| 51 | GACCAACAGCGCCCCCACCAGCAGCAGCACCAAGAAGACCCAGCTGCAGC | 100 |
| | . . . . . | |
| 739 | TGGAGCATTTACTGCTGGATTTACAGATGATTTTGAATGGAATTAATAAT | 788 |
| | \|\|\|\|\|\|\| \| \|\|\|\|\|\|\|\| \| \|\|\|\|\|\|\|\| \|\|\|\| \|\| \|\| \|\| \|\| | |
| 101 | TGGAGCACCTGCTGCTGGACCTGCAGATGATCCTGAACGGCATCAACAAC | 150 |
| | . . . . . | |
| 789 | TACAAGAATCCCAAACTCACCAGGATGCTCACATTTAAGTTTTACATGCC | 838 |
| | \|\|\|\|\|\|\|\| \|\|\|\|\| \|\| \|\|\| \| \|\|\|\|\| \|\| \|\| \|\|\|\|\| \|\|\|\|\|\|\|\| | |
| 151 | TACAAGAACCCCAAGCTGACCCGCATGCTGACCTTCAAGTTCTACATGCC | 200 |
| | . . . . . | |
| 839 | CAAGAAGGCCACAGAACTGAAACATCTTCAGTGTCTAGAAGAAGAACTCA | 888 |
| | \|\|\|\|\|\|\|\|\|\|\|\| \|\| \|\|\|\|\| \|\| \|\| \|\|\|\|\| \|\| \|\| \|\| \|\| \|\| \| | |
| 201 | CAAGAAGGCCACCGAGCTGAAGCACCTGCAGTGCCTGGAGGAGGAGCTGA | 250 |
| | . . . . . | |
| 889 | AACCTCTGGAGGAAGTGCTAAATTTAGCTCAAAGCAAAAACTTTCACTTA | 938 |
| | \| \|\| \|\|\|\|\|\|\|\| \|\|\|\|\| \|\| \| \|\| \|\| \|\|\|\|\| \|\|\|\|\| \|\|\| \| | |
| 251 | AGCCCCTGGAGGAGGTGCTGAACCTGGCCCAGAGCAAGAACTTCCACCTG | 300 |
| | . . . . . | |
| 939 | AGACCCAGGGACTTAATCAGCAATATCAACGTAATAGTTCTGGAACTAAA | 988 |
| | \| \|\|\| \| \|\|\| \| \|\|\|\|\|\|\|\| \|\|\|\|\|\|\|\| \|\| \|\| \|\|\|\|\| \|\| \|\| | |
| 301 | CGCCCCCGCGACCTGATCAGCAACATCAACGTGATCGTGCTGGAGCTGAA | 350 |
| | . . . . . | |
| 989 | GGGATCTGAAACAACATTCATGTGTGAATATGCTGATGAGACAGCAACCA | 1038 |
| | \|\|\| \|\| \|\| \|\| \|\|\|\|\|\|\|\| \|\| \|\| \|\| \|\| \|\|\|\|\| \|\| \|\|\|\| | |
| 351 | GGGCAGCGAGACCACCTTCATGTGCGAGTACGCCGACGAGACCGCCACCA | 400 |
| | . . . . . | |
| 1039 | TTGTAGAATTTCTGAACAGATGGATTACCTTTTGTCAAAGCATCATCTCA | 1088 |
| | \| \|\| \|\| \|\| \|\|\|\|\|\| \| \|\|\|\|\| \|\|\|\|\| \|\| \|\| \|\|\|\|\|\|\|\|\| | |
| 401 | TCGTGGAGTTCCTGAACCGCTGGATCACCTTCTGCCAGAGCATCATCAGC | 450 |
| | . | |
| 1089 | ACACTGACTTGA | 1100 |
| | \|\| \|\|\|\|\| \|\|\| | |
| 451 | ACCCTGACCTGA | 462 |

It was determined that both the natural coding sequence and the synthetic coding sequence are translated to form the identical polypeptide, the sequence of which is shown in Table II as the translation of the natural coding sequence (SEQ ID NO. 3).

TABLE II
TRANSLATION of WILD TYPE IL-2

| | |
|---|---|
| 1 | MYRMQLLSCI ALSLALVTNS APTSSSTKKT QLQLEHLLLD LQMILNGINN |
| 51 | YKNPKLTRML TFKFYMPKKA TELKHLQCLE EELKPLEEVL NLAQSKNFHL |
| 101 | RPRDLISNIN VIVLELKGSE TTFMCEYADE TATIVEFLNR WITFCQSIIS |
| 151 | TLT* |

VI. Administration

Administration as used herein refers to the route of introduction of the HGD composition into the body of an organism. Administration includes but is not limited to intravenous, intramuscular, systemic, subcutaneous, subdermal, topical, or oral methods of delivery. Administration can be directly to a target tissue or through systemic delivery.

In particular, the present invention can be used for administering nucleic acid for expression of specific nucleic acid sequences in cells. Routes of administration include intramuscular, aerosol, olfactory, oral, topical, systemic, ocular, intraperitoneal and/or intratracheal. A preferred method of administering HGD compositions is by oral delivery. Another preferred method of administration is by direct injection into the cells or by systemic intravenous injection.

The direct transfer of genes has proven to be very effective. Experiments show that administration by direct injection of DNA into joints and thyroid tissue results in expression of the gene in the area of injection. Injection of plasmids containing IL-1 genes into the spaces of the joints results in expression of the gene for prolonged periods of time. The injected DNA appears to persist in an unintegrated extrachromosomal state. This means of transfer is one of the preferred embodiments.

In addition, another means to administer the HGD compositions of the present invention is by using a dry powder form for inhalation. Furthermore, administration may also be through an aerosol composition or liquid form into a nebulizer mist and thereby inhaled.

The special delivery route of any selected vector construct will depend on the particular use for nucleic acid molecules associated with the HGD composition. In general, a specific delivery program for each HGD composition will focus on uptake with regard to the particular targeted tissue, followed by demonstration of efficacy. Uptake studies will include uptake assays to evaluate cellular uptake of the nucleic acid molecules and expression of the specific nucleic acid molecules of choice. Such assays will also determine the localization of the target nucleic acid molecules after uptake, and establishing the requirements for maintenance of steady-state concentrations of expressed protein. Efficacy and cytotoxicity is then tested. Toxicity will not only include cell viability but also cell function. Examples of these tests and assays are set forth herein.

The chosen method of delivery should result in cytoplasmic accumulation and optimal dosing. The dosage will depend upon the disease and the route of administration but should be between 0.1-1000 mg/kg of body weight/day. This level is readily determinable by standard methods. The level is variable depending on the optimal dosing. The duration of treatment will extend through the course of the disease symptoms, possibly continuously. The number of doses will depend upon disease delivery vehicle and efficacy data from clinical trials.

Establishing therapeutic levels of nucleic acid molecules or oligonucleotides within the cell may be dependent upon the rate of uptake and degradation of these molecules. Decreasing the degree of degradation can prolong the intracellular half-life of the nucleic acid molecules or oligonucleotides.

VII. Cell Transfection

One embodiment of the present invention includes cells transfected with nucleic acid molecules associated with the HGD compositions described above. Once the cells are transfected, the cells will express the protein, polypeptide, or RNA encoded for by the nucleic acid. Cells include, but are not limited to, liver, muscle, and skin. This description is not intended to be limiting in any manner.

The nucleic acid molecules comprising genetic material of interest may be positionally and sequentially oriented within the host or vectors such that the nucleic acid molecules can be transcribed into RNA and, when desired, be translated into proteins or polypeptides in the transfected cells. A variety of proteins and polypeptides can be expressed by the sequence in the nucleic acid cassette in the transfected cells. These products may function as intracellular or extracellular structural elements, ligands, hormones, neurotransmitters, growth regulating factors, apolipoproteins, enzymes, serum proteins, receptors, carriers for small molecular weight compounds, drugs, immunomodulators, oncogenes, tumor suppressors, toxins, tumor antigens, antigens, antisense inhibitors, triple strand forming inhibitors, ribozymes, or as a ligand recognizing specific structural determinants on cellular structures for the purpose of modifying their activity.

Transfection can be accomplished by in vivo or ex vivo techniques. One skilled in the art is familiar with such techniques for transfection. Transfection by ex vivo techniques includes co-transfecting the cells with nucleic acid molecules that harbor a selectable marker. This selectable marker can be used to select those cells which have become transfected. Selectable markers are well known to those who are skilled in the art.

For example, one approach to macromolecule delivery for hepatic diseases is to remove hepatocytes from an affected individual, genetically alter them in vitro, and re-implant them into a receptive locus. The ex vivo approach can include the steps of harvesting hepatocytes, cultivating the hepatocytes, transducing or transfecting the hepatocytes, and introducing the transfected hepatocytes into the affected individual.

The hepatocytes may be obtained in a variety of ways. They may be taken from the individual who is to be later injected with the hepatocytes that have been transfected or they can be collected from other sources, transfected and then injected into the individual of interest.

Once the ex vivo hepatocyte is collected, it may be transfected by contacting the hepatocytes with media that comprises a HGD composition and maintaining the cultured hepatocytes in the media for sufficient time and under conditions appropriate for uptake and transfection of the hepatocytes. The hepatocytes may then be introduced into an orthotopic location (i.e., the body of the liver or the portal vasculature) or heterotopic locations by injection of cell suspensions into tissues. One skilled in the art will recognize that the cell suspension may comprise salts, buffers, or nutrients to maintain viability of the cells as well as proteins to ensure cell stability, and factors to promote angiogenesis and growth of the implanted cells.

In an alternative method, harvested hepatocytes may be grown ex vivo on a matrix consisting of plastics, fibers or gelatinous materials which may be surgically implanted in an orthotopic or heterotopic location after transduction. This matrix may be impregnated with factors to promote angiogenesis and growth of the implanted cells. Cells can then be re-implanted. These examples are provided for illustrative purposes and are nonlimiting.

VIII. Direct Delivery of Macromolecules to the Liver

HGD compositions of the present invention can also be used in reversing or arresting the progression of disease involving the liver, such as liver cancer. One embodiment involves use of intravenous methods of administration for delivering nucleic acid molecules which encode a necessary molecule that may treat disease in the liver. HGD compositions which express a necessary protein or RNA can be directly injected into the liver or blood supply so as to travel directly to the liver.

IX. Direct Delivery of Macromolecules to Muscle

The muscular dystrophies are a group of diseases that result in abnormal muscle development, as a result of multiple factors. These diseases can be treated by using the direct delivery of genes with the HGD compositions of the present invention resulting in the production of normal gene product. Delivery to the muscle using methods of the present invention is performed to present genes that produce various antigens for vaccines against a multitude of infections of both viral, bacterial, and parasitic origin. The detrimental effects caused by aging can also be treated using the HGD-based compositions described herein. Since the injection of the growth hormone protein promotes growth and proliferation of muscle tissue, the growth hormone gene can be delivered to muscle, resulting in both muscle growth and development, which is decreased during the later portions of the aging process. Genes expressing other growth related factors can be delivered, such as Insulin Like Growth Factor-1 (IGF-1). Furthermore, any number of different genes may be delivered by this method to the muscle tissue.

IGF-1 can be used to deliver DNA to muscle, since it undergoes uptake into cells by receptor-mediated endocytosis. This polypeptide is 70 amino acids in length and is a member of the growth factor family and is structurally related to insulin. It is involved in the regulation of tissue growth and cellular differentiation effecting the proliferation and metabolic activities of a wide variety of cell types, since the polypeptide is specific for receptors on many types of tissue. As a result, the HGD compositions of the present invention can include IGF-1 as a ligand for tissue-specific nucleic acid delivery to muscle. The advantage of an IGF-1/nucleic acid molecule delivery system iis that the specificity and the efficiency of the delivery is greatly increased due to a great number of cells coming into contact with the ligand/composition with uptake through receptor-mediated endocytosis. Use of the HGD compositions that comprise the nucleic acid molecules of the invention and/or specific receptor and nuclear ligands described herein provides treatment of diseases and abnormalities that affect muscle tissues.

X. Direct Delivery of Macromolecules to Osteogenic Cells

One major disfunction associated with the aging process is osteoporosis, which is a decrease in overall bone mass and strength. The direct delivery of HGD compositions of the present invention can be utilized to deliver genes to cells that promote bone growth. Osteoblasts are the main bone forming cell in the body, but there are other cells that are capable of aiding in bone formation. The stromal cells of the bone marrow are the source of stem cells for osteoblasts. The stromal cells differentiate into a population of cells known as Inducible Osteoprogenitor Cells (IOPC), which under induction of growth factors, differentiate into Determined Osteoprogenitor Cells (DOPC). It is this population of cells that mature directly into bone producing cells. The IOPCs are also found in muscle and soft connective tissues. Another cell type involved in the bone formation process is the cartilage-producing cell known as a chondrocyte.

A factor identified to be involved in stimulating the IOPCs to differentiate is known as Bone Morphogenetic Protein (BMP). This 19,000 MW protein was first identified from demineralized bone. Another similar factor is Cartilage Induction Factor (CIF), which also functions to stimulate IOPCs to differentiate, thereby initiating cartilage formation, cartilage calcification, vascular invasion, resorption of calcified cartilage, and finally induction of new bone formation. Cartilage Induction Factor has been identified as being homologous to Transfecting Growth Factor β.

Since osteoblasts are involved in bone production, genes that enhance osteoblast activity can be delivered directly to these cells. Genes can also be delivered to the IOPCs and the chondrocytes, which can differentiate into osteoblasts, leading to bone formation. BMP and CIF are the ligands that can be used to deliver genes to these cells. Genes delivered to these cells promote bone formation or the proliferation of osteoblasts. The polypeptide, IGF-1 stimulates growth in hypophysectomized rats. This growth stimulation could be due to specific uptake of IGF-1 by osteoblasts or by the interaction of IGF-1 with chondrocytes, which results in the formation of osteoblasts. Other specific bone cell and growth factors can be used through the interaction with various cells involved in bone formation to promote osteogenesis.

Nonlimiting examples of growth factor genes that may induce a therapeutic effect where expressed in osteoblast cells are Insulin, Insulin-Like Growth Factor-1, Insulin-Like Growth Factor-2, Epidermal Growth Factor, Transfecting Growth Factor-α, Transfecting Growth Factor-β Platelet Derived Growth Factor, Acidic Fibroblast Growth Factor, Basic Fibroblast Growth Factor, Bone Derived Growth Factors, Bone Morphogenetic Protein, Cartilage Induction Factor, Estradiol, and Growth Hormone. All of these factors have been shown to induce a positive effect on the proliferation of osteoblasts, the related stem cells, and chondrocytes. As a result, BMP or CIF can be used as conjugates to deliver genes that express these growth factors to the target cells by the intravenous injection of HGD compositions of the present invention. Incorporating the nucleic acid molecules described above in the HGD compositions of the present invention, in conjunction with the use of specific ligands for the delivery of nucleic acid molecules to bone cells, may provide treatments for diseases and abnormalities that effect bone tissues.

XI. Direct Delivery of Macromolecules to Synoviocytes

The inflammatory attack upon joints in animal models and human diseases may be mediated, in part, by secretion of cytokines such as IL-1 and IL-6 which stimulate local inflammatory responses. Inflammatory reactions may be modified by local secretion of soluble fragments of the receptors for these ligands. The complex between ligands and soluble receptors may prevent ligands from binding to the receptors normally present on the surface of cells, thus preventing the stimulation of the inflammatory effect.

Therapy can consist of the construction of a DNA plasmid comprising the soluble form of receptors for appropriate cytokines (for example, IL-1), together with promoters capable of inducing high level expression in structures of the joint and composition. The plasmid can then be formulated into the composition comprising HGDs of the invention. The composition may be injected into effected joints where the secretion of an inhibitor for IL-1, such as a soluble IL-1 receptor or natural IL-1 inhibitor, modifies the local inflammatory response and resulting arthritis.

This method may be useful for treating episodes of arthritis which characterize many “autoimmune” or “collagen vascular”, diseases. This method can also prevent disabling injury of large joints by inflammatory arthritis.

The above-described compositions may also be useful for treating arthritis by incorporating a plasmid which encodes a gene that expresses a genetically modified steroid receptor. Current therapy for severe arthritis involves the administration of pharmacological agents including steroids to depress the inflammatory response. Steroids can be administered systemically or locally by direct injection into the joint space.

Steroids normally function by binding to receptors within the cytoplasm of cells. Formation of the steroid-receptor complex changes the structure of the receptor so that it becomes capable of translocating to the nucleus and binding to specific sequences within the genome of the cell and altering the expression of specific genes. Genetic modifications of the steroid receptor can enable this receptor to bind naturally occurring steroids with higher affinity, or bind non-natural, synthetic steroids, such as RU486. Other modifications can be made to create steroid receptor which is “constitutively active”, meaning that it is capable of binding to DNA and regulating gene expression in the absence of steroid in the same way that the natural steroid receptor regulates gene expression after treatment with natural or synthetic steroids.

Of particular importance is the effect of glucocorticoid steroids such as cortisone, hydrocortisone, prednisone, or dexamethasone which are the most important drugs available for the treatment of arthritis. One approach to arthritis treatment is to introduce a DNA plasmid in which the nucleic acid cassette expresses a genetically modified steroid receptor into cells of the joint, e.g.,. a genetically modified steroid receptor which mimics the effect of glucocorticoids but does not require the presence of glucocorticoids for effect. This receptor is termed the glucocortico-mimetic receptor. This effect is achieved by expression of a constitutively active steroid receptor within cells of the joint which contains the DNA binding domain of a glucocorticoid receptor. This induces the therapeutic effects of steroids without the systemic toxicity of these drugs.

Alternatively, steroid receptors which have a higher affinity for natural or synthetic glucocorticoids, such as RU486, can be introduced into the joint. These receptors exert an increased anti-inflammatory effect when stimulated by non-toxic concentrations of steroids or lower doses of pharmacologically administered steroids. Alternatively, constitution of a steroid receptor which is activated by a novel, normally-inert steroid enables the use of drugs which would affect only cells taking up this receptor. These strategies obtain a therapeutic effect from steroids on arthritis without the profound systemic complications associated with these drugs. Of particular importance is the ability to target these genes differentially to specific cell types (for example synovial cells versus lymphocytes) to affect the activity of these cells.

As described in U.S. Pat. No. 5,364,791 to Vegeto, et al., entitled “Progesterone Receptor Having C Terminal Hormone Binding Domain Truncations,” and U.S. application, Ser. No. 07/939,246, entitled “Mutated Steroid Hormone Receptors, Methods for Their Use and Molecular Switch for Gene Therapy,” Vegeto, et al., filed Sep. 2, 1992, both hereby incorporated by reference (including drawings), genetically modified receptors, such as the glucocortico-mimetic receptor, can be used to create novel steroid receptors including those with glucocortico-mimetic activity. The steroid receptor family of gene regulatory proteins is an ideal set of such molecules. These proteins are ligand activated transcription factors whose ligands can range from steroids to retinoids, fatty acids, vitamins, thyroid hormones and other presently unidentified small molecules. These compounds bind to receptors and either up-regulate or down-regulate transcription.

A preferred receptor of the present invention is a modified glucocorticoid receptor, i.e., a glucocorticoid-mimetic receptor. These modified receptors can bind various ligands whose structure differs from naturally occurring ligands, e.g., RU486. For example, small C-terminal alterations in amino acid sequence, including truncation, result in altered affinity and altered function of the ligand. By screening receptor mutants, receptors can be customized to respond to ligands which do not activate the host cells own receptors.

Methods and compositions of the present invention can also be used to treat arthritis when they comprise a gene encoding RNA that specifically binds and neutralizes the mRNA for prostaglandin synthase. Drugs which inhibit the enzyme prostaglandin synthase are important agents in the treatment of arthritis. This is due, in part, to the important role of certain prostaglandin in stimulating the local immune response. Salicylates are widely used drugs but can be administered in limited doses which are often inadequate for severe forms of arthritis.

Gene transfer using the present invention can be used to inhibit the action of prostaglandin synthase specifically in affected joints by the expression of an antisense RNA for prostaglandin synthase. The complex formed between the antisense RNA and mRNA for prostaglandin synthase may interfere with the proper processing and translation of this mRNA and lower the levels of this enzyme in treated cells. Alternatively RNA molecules may be used for forming a triple helix in regulatory regions of genes expressing enzymes required for prostaglandin synthesis. Alternatively, RNA molecules are identified which bind the active site of enzymes required for prostaglandin synthesis and inhibit this activity.

Alternatively, genes encoding enzymes which alter prostaglandin metabolism can be transferred into the joint. These have an important anti-inflammatory effect by altering the chemical composition or concentration of inflammatory prostaglandin.

Likewise, the present invention is useful for enhancing repair and regeneration of the joints. The regenerative capacity of the joint is limited by the fact that chondrocytes are not capable of remodeling and repairing cartilaginous tissues such as tendons and cartilage. Further, collagen which is produced in response to injury is of a different type lacking the tensile strength of normal collagen. Further, the injury collagen is not remodeled effectively by available collagenase. In addition, inappropriate expression of certain metalloproteinases is a component in the destruction of the joint.

Gene transfer using promoters specific to chondrocytes (i.e., collagen promoters) is used to express different collagens or appropriate collagenase for the purpose of improving the restoration of function in the joints and prevent scar formation.

Compositions of the invention can facilitate the uptake of nucleic acid molecules into chondrocytes and synovial cells by direct injection of the compositions into the effected joint space. Further, the nucleic acid molecules may permeate into the environment of the joint where they are taken up by fibroblasts, myoblasts, and other constituents of periarticular tissue.

XII. Direct Delivery of Macromolecules to the Lungs

HGD compositions of the present invention can be utilized in methods for reversing or arresting the progression of disease involving the lungs, such as lung cancer. One embodiment involves using intravenous methods of administration for delivering nucleic acid molecules that encode a molecule that has a therapeutic effect on lung disease. HGD compositions which express a protein or RNA can be directly injected into the lungs or blood supply so as to travel directly to the lungs. Furthermore, the use of an aerosol or a liquid in a nebulizer mist can also be used to administer the desired nucleic acid to the lungs. A dry powder form of the compositions can also be used to treat disease in the lung. The dry powder form of the compositions is delivered by inhalation. These treatments can be used to control or suppress lung cancer or other lung diseases by expression of a particular protein encoded by the nucleic acid molecules. Importantly, HGDs of the invention can be fused to IL-12 or other lung specific targeting molecules for specific delivery of a complex of the invention to the lungs.

Additional organs, tissues, cavities, cell or cells, and spaces for the administration of the molecules mentioned herein may be found in “Nucleic Acid Transporters for Delivery of Nucleic Acids into a Cell”; Smith et al., U.S. patent application Ser. No. 08/484,777, filed Dec. 18, 1995, incorporated herein by reference in its entirety including any drawings.

XIII. Formulations for in Vivo Deliverv

While expression systems such as those described above provide the potential for RNA transcription and polypeptide expression when delivered to an appropriate location, it is beneficial to provide the expression system construct(s) in a delivery system which can assist both the delivery and the cellular uptake of the construct. Thus, the invention also provides particular formulations which include one or more expression system constructs (e.g., DNA plasmids as described above), a cationic lipid, a co-lipid (preferably a neutral co-lipid), and a carbohydrate or other osmolyte (e.g., salts) to make the formulation iso-osmotic and isotonic. Formulation methods are also provided herein, for example.

In addition to the lipids and lipid combinations described herein for exemplary formulation embodiments, other lipids may be selected. Examples of such lipids are described in Gao & Huang, 1995, Gene Therapy 2:710-722 and are summarized in Table III.

| | | | Transfection | |
|---|---|---|---|---|
| | | | Activity | |
| Cationic | | | In | In |
| Liposome | Compositon | Manufacturer | vitro | vivo |
| A: | | | | |
| Commercialized | | | | |
| Lipofectin | DOTMA/DOPE = | GIBCO BRL | +++ | ++ |
| | 1:1 (w/w) | | | |
| DOTAP | DOTAP | Boehringer | +++ | ++ |
| | | Mannheim | | |
| TransfectAse | DDAB/DOPE = | GIBCO BRL | +++ | NA |
| | 1:3 (m/m) | | | |
| LipofectAMINE | DOSPA/DOPE = | GIBCO BRL | ++++ | NA |
| | 3:1 (w/w) | | | |
| Transfectam | DOGS | Promega | ++++ | +++ |
| B. Not | | | | |
| commercialized | | | | |
| CTAB | CTAB/DOPE = | | ++ | NA |
| | 1:4 (m/m) | | | |
| C₁₃CluPhCnNa | C₁₃GluPhCnN? | | +++ | NA |
| C₁₃GluCnN? | C₁₃GluCnN? | | +++ | NA |
| Lipopolylysine | Lipopolylyaine/ | | +++ | NA |
| | DOPE = 1:8 (m/m) | | | |
| Cationic | Cationic chol/ | | YES | +++ |
| cholesterols | DOPE = 1:1 (m/m) | | | |
| DC-chol | DC-chol/DOPE = | | +++ | +++ |
| | 3:2 (m/m) | | | |
| DMRIE | DMRIE/ | | +++ | +++ |
| | DOPE = 1:1 (m/m) | | | |
| DOTMA/chol | DOTXA/ | | +++ | NA |
| | cholesterol = | | | |
| | 1:1 (m/m) | | | |
| Lysyl-PE | Lysyl-PE/β-alanyl | | +++ | NA |
| | cholesterol = | | | |
| | 1:1 (m/m) | | | |

In compositions in which lipids, such as DOTMA and cholesterol, are used, preferably, though not necessarily, the cationic lipid and the neutral co-lipid may be formed into liposomes, such as by forcing the lipid and aqueous solution through a membrane with pores of a desired size or by microfluidization. The liposomes may be combined with the DNA to form a DNA/lipid complex, which can then be administered to a mammal by a delivery method appropriate to the desired delivery site.

Formation of liposomes by microfluidization provides liposomes of discrete size, i.e., the size distribution is narrower than other preparation methods tested. This preparation method can, as an example, be performed as follows. For the exemplary DOTMA/cholesterol formulations, the liposomes are composed of the cationic lipid DOTMA (1,2-di-O-octadecenyl-3-trimethylammonium-propane, chloride salt) and the uncharged co-lipid cholesterol mixed to give a 50:50 mole percent ratio. DOTMA and cholesterol may be mixed as a 50:50 mole percent ratio, and then lyophilized.

The lyophilized material can be analyzed with respect to identity and composition, then rehydrated with sterile water for irrigation USP to allow liposome formation. The rehydrated material is microfluidized to generate a homogenous population of small liposomes. The microfluidized material is then 0.2 micron-filtered and analyzed with respect to composition, particle size, and sterility.

During the process of microfluidization, a source of ultrapure nitrogen gas (Air Liquide) is used to drive the air driven pressure system of the microfluidizer M110S (Microfluidics Corporation) unit, however, other microfluidizers could also be used. The lyophilized lipid mixture is allowed to come to room temperature, then sterile water for irrigation U.S.P., approximately 200 mL, is added. The mixture is allowed to rehydrate for approximately 1.5 hours before any further processing. Once rehydration is completed and a translucent homogeneous suspension is obtained, the liposomes are microfluidized. For this, a pressure of approximately 50,000 psi is applied to the microfluidizer unit and the sample is cycled through the chamber at least 10 times, which in turn produces a population of vesicles of reduced size. Following microfluidization, a sample is collected for an in-process particle check. After a predetermined particle size is achieved, the material can be removed and filtered through a 0.2 micron filter. At this point, the material may be diluted with sterile water for irrigation U.S.P. to achieve a desired concentration. The liposome solution may be vialed under an overlay of Argon gas.

A description of the use of liposomes for gene transfer is provided in Szala et al., 1996, Gene Therapy 3:1026-1031. In this report, cationic liposomes using DC-Cholesterol/DOPE and DDAB/DOPE were used to transfer E. coli cytosine deaminase gene into melanoma tumors by direct injection.

As described below, the selection of non-DNA formulation components, the diameter and size distribution of the liposomes, and the DNA/HGD charge ratio can be significant parameters in determining the resulting level of expression.

An additional significant factor relating to the plasmid construct is the percentage of plasmids which are in a supercoiled (SC) form rather than the open circular (OC) form.

The in vivo effects of such formulations is particularly notable in comparison with the in vivo effects of an alternative formulation which can produce even higher levels of encoded product, i.e., a DNA:PVP formulation.

### EXAMPLES

### EXAMPLE 1 SYNTHETIC PROCEDURES FOR HYDROPHOBIC GLYCOSYLAMINE DERIVATIVES

The following general schemes were utilized to prepare 1-substituted and 3,4-substituted glycosylamine derivatives, respectively.

Preparation of 1-substituted Glycosylamine Derivatives

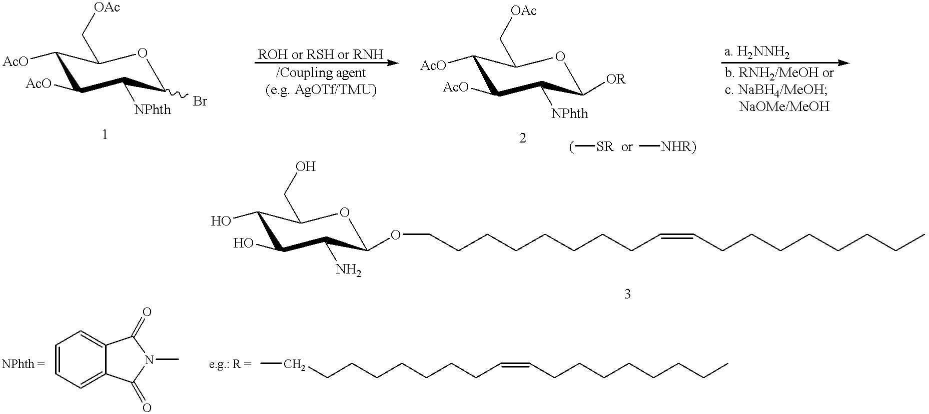

Preparation of 3-, 4-, and 3,4-substituted glycosylamine derivatives:

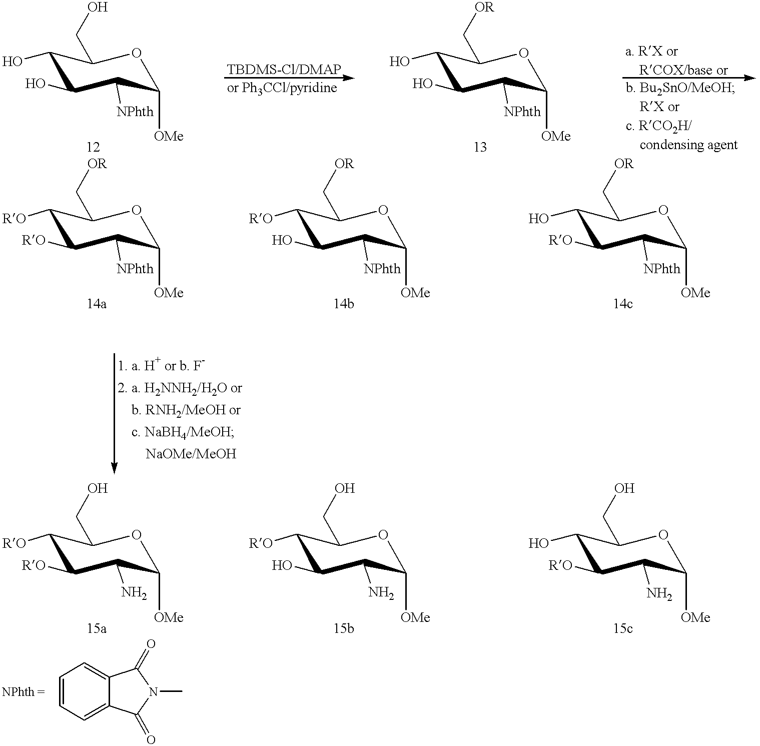

The following synthetic sequence was utilized to prepare a 1-modified cationic glucosamine derivative, 1-mono-oleyl-β-D-glucosamine. Bromo-3,4,6-tri-O-acetyl-2-deoxy-2-phthalimido-α-D-glucose (0.5 g, 1.06 mmol from Toronto Research Chemicals) was treated with oleyl alcohol (0.67 mL, 2.12 mmol from Sigma, St. Louis, Mo.) in the presence of silver trifluoromethanesulfonate (0.55 g, 2.12 mmol), symcollidine (0.14 mL, 1.06 mmol) and powdered 4Å molecular sieves (0.75 g) in dry methylene chloride (10 mL) for one hour under an argon atmosphere. The solids were then removed by filtration and the solution was evaporated to an oil which was chromatographed on a column of silica gel eluted with hexanes:ethyl acetate. The desired product, oleyl-3,4,6-tri-O-acetyl-2-deoxy-2-phthalimido-β-D-glucoside, was characterized by ¹H—NMR and FAB-MS. Removal of the acetyl groups was accomplished by treatment of the fully protected compound (0.5 g, 0.7 mmol) with methanolic ammonia (10 mL of 2 M) followed by evaporation under pressure. Removal of the phthalimido moiety was then accomplished by redissolving the deacylated material (0.15 g, 0.3 mmol) in ethanol (2 mL) followed by addition of hydrazine hydrate (0.07 mL, 1.2 mmol) and workup using techniques well known to a person of ordinary skill in the art. The final product 1-mono-oleyl-β-D-glucosamine was purified by passing the crude mixture down a column of silica gel eluted with chloroform:methanol:acetic acid:water (90:10:0.8:0.4). The product was analyzed by ¹H—NMR and FAB-MS.

The following synthetic sequence outlines the preparation of 1-mono-oleyl-β-D-glucosamine (MOG). The final product was analyzed by ¹H—NMR and FAB-MS.

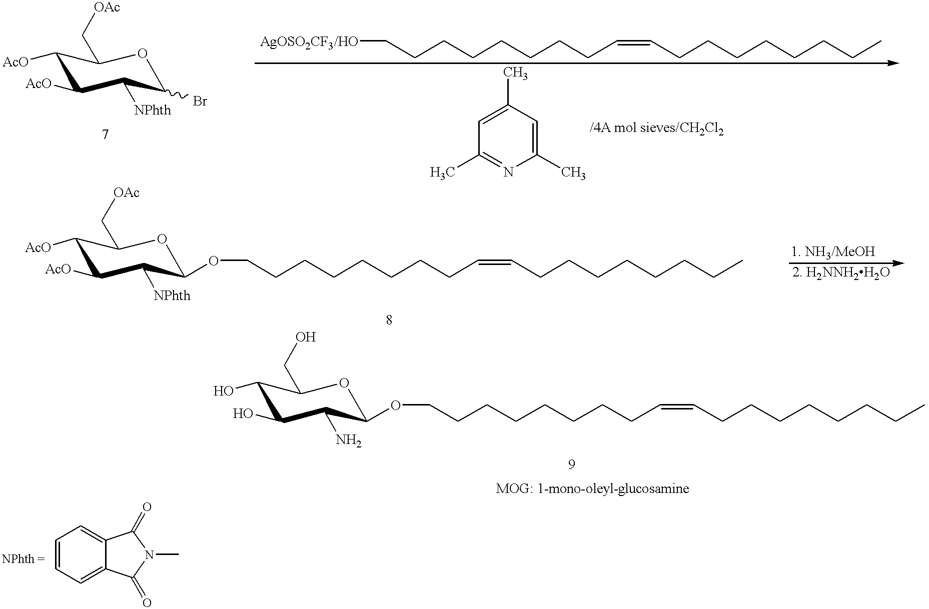

The following synthetic sequence outlines the preparation of 1-mono-palmityl-β-D-glucosamine (MPG). The reaction scheme is analogous to that for the oleyl derivative with hexadecanol used in place of oleyl alcohol in the first step. The final product was analyzed by ¹H—NMR and FAB-MS.

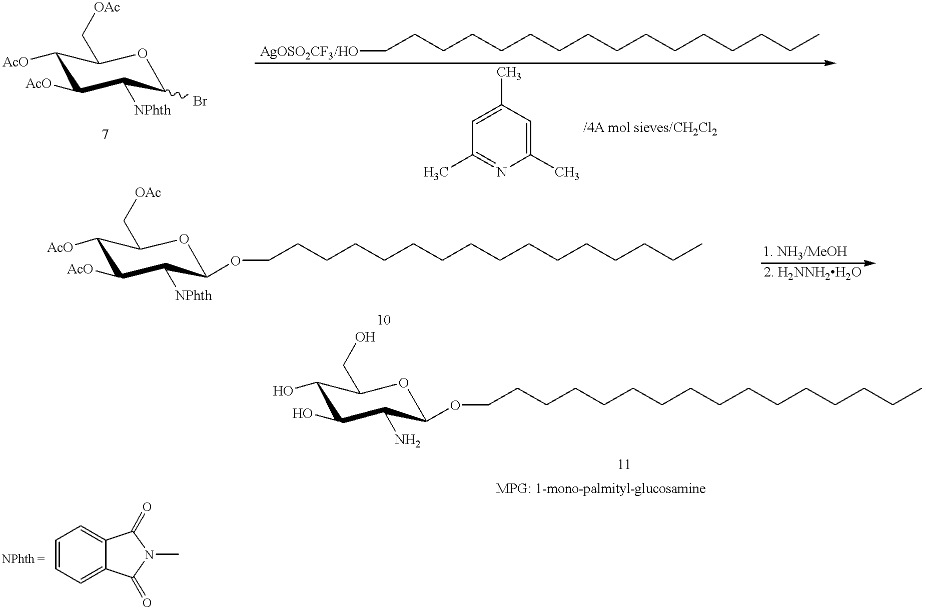

### EXAMPLE 2 FORMULATION PROCEDURE FOR COMPOSITIONS OF THE INVENTION

Two representative glycosylamine lipids, MOG and MPG, were examined for their ability to condense and deliver DNA. MOG and MPG were mixed with co-lipids such as DOPE and cholesterol and mixed with DNA plasmids. These compositions were formulated into liposomes or micelles using a solvent evaporation method. This method involved mixing the HGD and DOPE (or cholesterol) in equimolar proportions in chloroform or chloroform/methanol solutions and then evaporated into a thin film. The film was hydrated by the addition of water to form a cloudy suspension at room temperature or 50° C., depending upon the composition of the lipid chain. The suspension was then either sonicated in a bath sonicator for several minutes and/or extruded through a membrane to yield the desired particle size. This preparation was then mixed with DNA plasmid at various charge ratios.

The resulting particles were analyzed for particle size, zeta potential, and electrophoretic mobility. When formulated with a co-lipid, the HGDs of the invention condensed DNA plasmids. The DNA plasmid condensation was evident by light scattering measurements, which indicated that the particles had an average diameter between 150 nanometers and 300 nanometers. The HGDs of the invention formed condensed DNA/HGD complexes with minimum diameters occurring when the −/+ charge ratio was between 1:1 and 1:3. The in vitro transfection efficiency and in vivo anti-tumor efficacy were determined for these formulated compositions in the following examples.

### EXAMPLE 3 TRANSFECTION EFFICIENCIES OF HYDROPHOBIC GLYCOSYLAMINE COMPOSITIONS OF THE INVENTION

HGDs of the invention formed DNA/HGD complexes that efficiently delivered DNA plasmids to cells. The transfection efficiency was measured after compositions comprising HGDs of the invention, 0.2 μg of DNA plasmids comprising a gene encoding IL-2, and either cholesterol or DOPE as a co-lipid were incubated with cells for 48 hours. The amount of IL-2 expressed by cells administered the compositions was quantified using by an assay disclosed in U.S. application Ser. No. 60/039,709, Raiston et al., “IL-2 Gene Expression and Delivery Systems and Uses,” filed Feb. 10, 1997, incorportated herein by reference in its entirety including any figures, tables, and drawings.

IL-2 was not significantly expressed by cells when the composition comprised the glycosylamine derivative MOG and had a −/+ charge ratio of 1:0.5. However, IL-2 was significantly expressed by cells administered a composition comprising MOG and having a 1:3 −/+ charge ratio. Specifically, the IL-2 expression for compositions comprising MOG and having a 1:3 charge ratio was as large as the amount of IL-2 expressed by cells administered liposomes comprising an efficient delivery lipid, DOTMA.

### EXAMPLE 4

### CELL TOXICITY DETERMINATIONS FOR COMPOSITIONS COMPRISING GLYCOSYLAMINE DERIVATIVES

Compositions comprising HGDs of the invention, plasmid DNA, and a co-lipid such as DOPE or cholesterol were not appreciably toxic to cells. Toxicity measurements were based upon total cellular protein synthesis (Bio-Rad Protein Assay) or cell proliferation (Cell-Proliferation Kit-XTT, Boehringer Mannheim).

Cells administered compositions comprising MOG and DOPE in a 1:1 ratio produced between 65% to 80% of the protein normally expressed by cells when the −/+ charge ratio was between 1:1 and 1:6. Cells produced between 65% to 70% of the protein normally expressed by cells when they were administered compositions comprising MPG and DOPE in a 1:1 ratio where the −/+ charge ratio was between 1:1 and 1:9. Cells produced between 50% to 65% of the protein normally expressed by cells administered compositions comprising MPG and DOPE in a 1:1 ratio where the −/+ charge ratio was between 1:1 and 1:9. Typically, a lower −/+ charge ratio corresponded to higher protein synthesis and thereby lower cell toxicity.

An advantage of the HGDs of the invention was evident by the cell toxicity studies. Specifically, cells administered liposome compositions comprising MOG or MPG were healthier than cells administered liposome compositions comprising a commercially available lipid, LipofectAMINE®. In fact, expression and recovery of total protein improved from 20% of normal protein levels to more than 75% of normal protein levels when a composition comprising MOG and DOPE was administered to cells instead of a composition comprising LipofectAMINE®. Thus, HGDs of the invention provide for delivery compositions that are less toxic to cells than compositions comprising commercially available lipids.

### EXAMPLE 5: DELIVERY OF HYDROPHOBIC GLYCOSYLAMINE DERIVATIVES TO TUMORS

A variety of HGD/DNA complexes were injected into SCCVII tumors. DNA plasmids harboring a CAT reporter gene were utilized for these demonstrations. Complexes comprising MOG and DNA plasmid induced the highest expression of the CAT reporter gene of any of the lipophilic molecule/DNA complexes tested. Other lipophilic molecules included DOTMA complexed with cholesterol, DOTIM complexed with DOPE and cholesterol, MMCE complexed with DOPE and cholesterol and DiP-PE, EPMC complexed with DOPE and cholesterol. All complexes comprised 10% lactose.

### EXAMPLE 6

### IN VIVO ANTI-TUMOR EFFICACY OF COMPOSITIONS COMPRISING HYDROPHOBIC GLYCOSYLAMINE DERIVATIVES OF THE INVENTION

Compositions comprising HGDs of the invention, plasmid DNA comprising an IL-2 gene, and DOPE efficaciously retarded the growth of tumors in mice. These model-tumor studies involved implanting a tumor into mice, administering a composition of the invention directly into the tumor, and monitoring the growth of tumors in the mice. A protocol for such a procedure is taught in U.S. application Ser. No. 60/039,709, Raiston et al., “IL-2 Gene Expression and Delivery Systems and Uses,” filed Feb. 10, 1997, incorportated herein by reference in its entirety including any figures, tables, and drawings.

These studies indicated that the compositions comprising HGDs of the invention, specifically MOG and MPG, significantly reduced the growth of tumors in mice by efficiently delivering plasmid DNA that expressed human IL-2 in cells. For example, a composition comprising MOG, plasmid DNA, and DOPE in a 1:2 −/+ charge ratio reduced the growth rates of tumors after nine days by more than 30%. Specifically, the size of tumors for untreated mice was on average 600 cubic millimeters and the size of tumors after administering the MOG, DNA, DOPE composition was on average less than 400 cubic millimeters. In addition, a composition comprising MPG, plasmid DNA, and DOPE in a 1:2 −/+ charge ratio reduced the growth rates of tumors after thirteen days by more than 25%. Specifically, the size of tumors for untreated mice was on average approximately 1600 cubic millimeters and the size of tumors after administering the MPG, DNA, DOPE composition was on average less than 1200 cubic millimeters.

Therefore, the in vivo mouse xenograft studies indicated that compositions comprising HGDs of the invention, plasmid DNA comprising an IL-2 gene, and a co-lipid efficaciously retarded the growth of tumors.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are within the following claims.

What is claimed is:

1. A compound comprising a glycosyl moiety having a nitrogen-based substituent linked to a carbon atom within said glycosyl moiety,
wherein said nitrogen-based substituent is selected from the group consisting of —NH₂, —N⁺(CH₃)₃, —(CH₂)ₙ—N (R₁₀)₃, and —NH—C (N⁺H₂) —NH₂, and
wherein substituents linked to other carbon atoms within said glycosyl moiety are independently selected from the group consisting of hydrogen, -alkyl, —O— alkyl, —O—C (O) -alkyl, —O—CH₂—CH₂ (O—C (O) —R₆) —CH₂ (O—C (O) —R₇), —O—CH₂—CH₂ (OR₆) —CH₂ (OR₇) , —CH₂—CH₂ (R₆) —CH₂(R₇) , —O—(CH₂)ₘ-cholesterol, polyethylene glycol, —O—(CH₂)ₙ—N (R₈)₃, —NH₂, —N⁺(CH₃)₃, —(CH₂)ₙ—N (R₉)₃, and —(CH₂)—OR₁₀,
wherein R₆, R₇, R₈, R₉, and R₁₀ are independently selected from the group consisting of hydrogen, methyl, and alkyl,
wherein m is selected from the group consisting of 0, 1, 2, 3, 4, and 5, and
wherein n is selected from the group consisting of 1, 2, 3, 4, and 5.

2. A compound of claim 1 having a structure set forth in formula I: 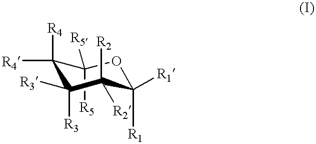
wherein said R₁ and R₁′ are independently selected from the group consisting of hydrogen, —OH, —OCH₃, -alkyl, —O— alkyl, —O—C(O)-alkyl, —O—CH₂—CH₂ (O—C (O) —R₆) —CH₂ (O—C (O) —R₇) , —O—CH₂—CH₂ (OR₆) —CH₂ (OR₇) , —O—CH₂—CH₂ (R₆) —CH₂ (R₇) , —O—(CH₂)ₘ-cholesterol, polyethylene glycol, —O—(CH₂)ₘ—N (R₈)₃, —NH₂, —N⁺(CH₃)₃, —(CH₂)ₙ—N (R₉)₃, and —(CH₂)—OR₁₀, wherein R₆, R₇, R₈, R₉, and R₁₀ are independently selected from the group consisting of hydrogen, methyl, and alkyl, and wherein m is selected from the group consisting of 0, 1, 2, 3, 4, and 5, and wherein n is selected from the group consisting of 1, 2, 3, 4, and 5;
wherein R₂ and R₂′ are independently selected from the group consisting of hydrogen, —NH₂, —N⁺(CH₃)₃, —(CH₂)ₙ—N (R₁)₃, and —NH—C (N⁺H₂) —NH₂, wherein said R₁₁ is selected from the group consisting of hydrogen, methyl, and alkyl; and
wherein R₃, R₃′, R₄, R₄′, R₅ and R₅′ are independently selected from the group consisting of hydrogen, —OH, —OCH₃, -alkyl, —O— alkyl, —O—C(O)-alkyl, —O—CH₂—CH₂ (O—C (O) —R₆) —CH₂ (O—C (O) —R₇), —O—CH₂—CH₂ (OR₆) —CH₂ (OR₇) , —O—CH₂—CH₂(R₆) —CH₂ (R₇) —O—(CH₂)ₘ-cholesterol, polyethylene glycol, —O—(CH₂)ₙ—N (R₈)₃, —NH₂, —N⁺(CH₃)₃, —(CH₂)—N (R₉)₃, and —(CH₂)—OR₁₀ wherein R₆, R_{7,} R₈, R₉, and Rlo are independently selected from the group consisting of hydrogen, methyl, and alkyl, and wherein m is selected from the group consisting of 0, 1, 2, 3, 4, and 5, and wherein n is selected from the group consisting of 1, 2, 3, 4, and 5;
provided that R₅′ is not —CH₂—O—C(O)-(CH₂) ₁₄CH₃ when R₃′ and R₄′ are —OH, R₂′ is —NH₂, and R₁′ is —OCH₃; and
provided that R₅′ is not —CH₂—O—C(O)-(CH₂)ₚCH₃, wherein p is selected from the group consisting of 10, 12, 14, or 16, when R₃′ is identical to R₅′, R₄′ is —OH, R₂′ is —NH₂, and R₁′ is —OCH₃.

3. The compound of claim 2, wherein R₂ and R₂′ are independently selected from the group consisting of hydrogen, —NH₂, —N⁺(CH₃)₃, and —NH—C (N⁺H₂) —NH₂—.

4. The compound of claim 3, wherein R₃, R₃′, R₄, R₄′, R₅, and R₅′ are independently selected from the group consisting of hydrogen, —OH, —O—C(O)-alkyl, —O— alkyl, -alkyl, and —(CH₂)—OH.

5. The compound of claim 4, wherein R₁ and R₁′ are independently selected from the group consisting of hydrogen, —OCH₃, -alkyl, —O— alkyl, —O—C (O) -alkyl, —O—CH₂—CH₂ (alkyl) —CH₂ (alkyl), —O—CH₂—CH₂ (O-alkyl) —CH₂ (O-alkyl), —O—CH₂—CH₂ (O—C (O) -alkyl) —CH₂ (O—C (O) -alkyl), —O—(CH₂)ₘ-cholesterol, —O—(CH₂),—NH₂, and —O—(CH₂)ₙ—N⁺(CH₃)₃, wherein m is selected from the group consisting of 0, 1, 2, 3, 4, and 5, and wherein n is selected from the group consisting of 1, 2, 3, 4, and 5.

6. The compound of claim 5, wherein said alkyl moiety is a straight chain hydrocarbon moiety having 14, 16, or 18 carbon atoms and 0, 1, 2, or 3 unsaturations.

7. The compound of claim 6 having the structure set forth in formula (II):

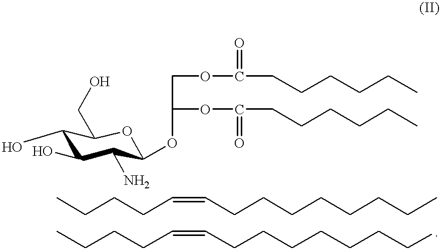

8. The compound of claim 6 having the structure set forth in formula (III):

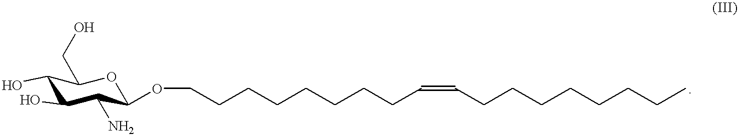

9. The compound of claim 6 having the structure set forth in formula (IV):

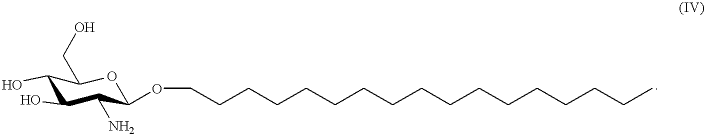

10. A composition for delivery one or more macromolecules into one or more cells, comprising:
(a) a compound comprising a glycosyl moiety having a nitrogen-based substituent linked to a carbon atom within said glycosyl moiety, wherein said nitrogen-based substituent is selected from the group consisting of —NH₂, —N⁺(CH₃)₃, —(CH₂)ₙ—N (R₁₀)₃, and —NH—C (N⁺H₂) —NH₂, and wherein substituents linked to other carbon atoms within said glycosyl moiety are independently selected from the group consisting of hydrogen, -alkyl, —O— alkyl, —O—C (O) -alkyl, —O—CH₂—CH₂ (O—C (O) —R₆) —CH₂ (O—C (O) —R₇), —O—CH₂—CH₂ (OR₆) —CH₂ (OR₇), —O—CH₂—CH₂ (R₆) —CH₂ (R₇), —O—(CH₂)ₘ-cholesterol, polyethylene glycol, —O—(CH₂)ₙ—N (R₈)₃, —NH₂, —N⁺(CH₃)₃, —(CH₂)ₙ—N (R₉)₃, and —(CH₂)—OR₁₀ wherein R_{6,} R₇, R₈, R₉, and R₁₀ are independently selected from the group consisting of hydrogen, methyl, and alkyl, and wherein m is selected from the group consisting of 0, 1, 2, 3, 4, and 5, and wherein n is selected from the group consisting of 1, 2, 3, 4, and 5; and
(b) said macromolecule or macromolecules.

11. A composition for delivering one or more macromolecules into one or more cells, comprising:
(a) a compound having a structure set forth in formula (I): 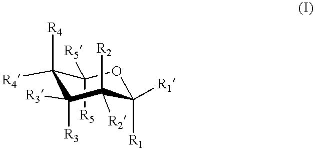
wherein R₁ and R₁′ are independently selected from the group consisting of hydrogen, —OH, —OCH₃, -alkyl, —O— alkyl, —O—C(O)-alkyl, —O—CH₂—CH₂(O—C(O)—R₆)—CH₂ (O—C (O) —R₇), —O—CH₂—CH₂ (OR₆) —CH₂ (OR₇), —O—CH₂—CH₂ (R₆) —CH₂ (R₇) , —O—(CH₂) m-cholesterol, —O—(CH₂)ₙ—N (R₈)₃, —NH₂, —N⁺(CH₃)₃, —(CH₂)ₙ—N (R₉)₃, and —(CH₂) —R₁₀, wherein R₆, R₇, R₈, R₉, and R₁₀ are independently selected from the group consisting of hydrogen, methyl, and alkyl, and wherein m is selected from the group consisting of 0, 1, 2, 3, 4, and 5, and wherein n is selected from the group consisting of 1, 2, 3, 4, and 5;
wherein R₂ and R₂′ are independently selected from the group consisting of hydrogen, —NH₂, —N⁺(CH₃) ₃, —(CH₂)ₙ—N (R₁₁)₃, and —NH—C (N⁺H₂) —NH₂, wherein said R₁₁ is selected from the group consisting of hydrogen, methyl, and alkyl; and
wherein R₃, R₃′, R₄, R₄′, R₅, and R₅′ are independently selected from the group consisting of hydrogen, —OH, —OCH₃, -alkyl, —O— alkyl, —O—C(O)-alkyl, —O—CH₂—CH₂ (O—C (O) —R₆) —CH₂ (O—C (O) —R₇), —O—CH₂—CH₂ (OR₆) —CH₂ (OR₇), —O—CH₂—CH₂ (R₆) —CH₂ (R₇), —O—(CH₂)ₘ-cholesterol, —O—(CH₂)ₙ—N(R₈)₃, —NH₂, —N⁺(CH₃)₃, —(CH₂)ₙ—N (R₉)₃, and —(CH₂)—OR₁,o wherein R₆, R₇, R₈, R₉, R₁₀ are independently selected from the group consisting of hydrogen, methyl, and alkyl, and wherein m is selected from the group consisting of 0, 1, 2, 3, 4, and 5, and wherein n is selected from the group consisting of 1, 2, 3, 4, and 5; and
(b) said macromolecule or macromolecules.

12. The composition of claim 11, wherein R₂ and R₂′ are independently selected from the group consisting of —NH₂, —N⁺(CH₃)₃, and —NH—C(N⁺H₂)—NH₂.

13. The composition of claim 12, wherein said R₃, R₃′, R₄, R₄′, R₅, and R₅′ are independently selected from the group consisting of hydrogen, —OH, —O—C(O)-alkyl, —O— alkyl, and -alkyl, —(CH₂)—OH.

14. The composition of claim 13, wherein said R₁ and R₁′ are independently selected from the group consisting of —OCH₃, -alkyl, —O— alkyl, —O—C(O)-alkyl, —O—CH₂—CH₂ (alkyl) —CH₂ (alkyl) —O—CH₂—CH₂ (O-alkyl) —CH₂ (O-alkyl), —O—CH₂—CH₂ (O—C (O) -alkyl) —CH₂ (O—C (O) -alkyl), —O—(CH₂)ₘ-cholesterol, —O—(CH₂),—NH₂, and —O—(CH₂)ₙ—N⁺(CH₃)₃, wherein m is selected from the group consisting of 0, 1, 2, 3, 4, and 5, and wherein n is selected from the group consisting of 1, 2, 3, 4, and 5.

15. The composition of claim 14, wherein said alkyl moiety is a straight chain hydrocarbon moiety having 14, 16, or 18 carbon atoms and 0, 1, 2, or 3 unsaturations.

16. The composition of claim 15, wherein said compound has the structure set forth in formula (II):

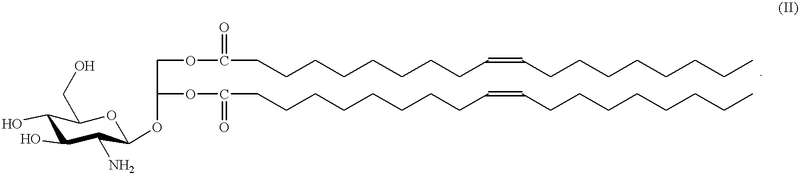

17. The composition of claim 15, wherein said compound has the structure set forth in formula (III):

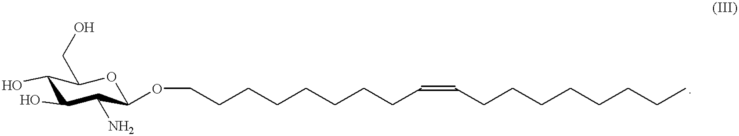

18. The composition of claim 15, wherein said compound has the structure set forth in formula (IV):

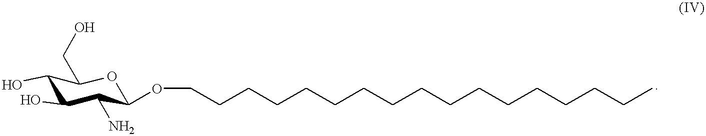

19. The composition of claim 11, wherein said macromolecule is an anionic molecule.

20. The composition of claim 19, wherein said anionic molecule is selected from the group consisting of a polynucleotide molecule, a DNA molecule, a RNA molecule, and a nucleotide analog molecule.

21. The composition of claim 20, wherein said DNA molecule is a plasmid molecule comprising at least one element for polypeptide expression in one or more eukaryotic cells.

22. The composition of claim 21, wherein said plasmid molecule further comprises a gene encoding IL-2.

23. The composition of claim 11, further comprising at least one co-lipid.

24. The composition of claim 23, wherein said co-lipid is DOPE.

25. The composition of claim 23, wherein said co-lipid is cholesterol.

26. The composition of claim 11, further comprising a cryoprotectant.

27. The composition of claim 26, wherein said cryoprotectant is PVP.

28. The composition of claim 11, wherein said composition is capable of forming liposomes.

29. The composition of claim 11 having an effective diameter between 100 nanometers and 300 nanometers.

30. The composition of claim 11 having a −/+ charge ratio selected from the group consisting of 1:0.5, 1:1, 1:2, 1:3, 1:4, 1:5, 1:6, and 1:9.

31. A method for delivering macromolecules to cells of a mammal, comprising the step of administering a composition of any one of claims 11-30 to said cells.

32. The method of claim 31, wherein said composition is administered to said cells in vitro.

33. The method of claim 31, wherein said composition is administered to said cells in vivo.

34. The method of claim 31, wherein said administration results in IL-2 expression in said cells.

35. The method of claim 31, wherein said composition is administered by a technique selected from the group consisting of direct injection to a tissue, parenteral injection, intravenous injection, oral administration, and administration by inhalation.

36. A method for synthesizing a compound of claim 2, comprising the steps of:
(a) reacting a first reactant of formula (V): 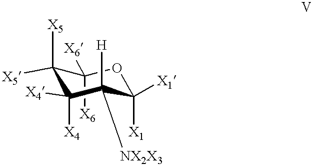
with a second reactant,
wherein X₁ and X₁′ are independently selected from the group consisting of hydrogen, halogen atom, and an activatable moiety; X₂ and X₃ are independently selected from the group consisting of a protecting moiety, hydrogen, halogen, or any activatable moiety; and X₄, X₄′, X₅, X₅′, X₆ and X₆′ are independently selected from the group consisting of hydrogen, —O—acetyl, —OH, —CH₂—O—acetyl, —CH₂—OH, and —O— alkyl;
wherein said second reactant is selected from the group consisting of HOCH₃, HO-alkyl, HO—C(O)-alkyl, HO—CH₂—CH₂ (O—C (O)—R₆) —CH₂ (O—C (O) —R₇), HO—CH₂—CH₂ (OR₆) —CH₂ (OR₇) , HO—CH₂—CH₂ (R₆) —CH₂(R₇) HO—(CH₂)ₘ-cholesterol, and HO—(CH₂)ₙ—N(R₈)₃, wherein R₆, R₇, R₈, and R₉are independently selected from the group consisting of hydrogen, methyl, and alkyl, and wherein m is selected from the group consisting of 0, 1, 2, 3, 4, and 5, and wherein n is selected from the group consisting of 1, 2, 3, 4, and 5;
(b) reacting the product of step (a) with a reducing agent; and
(c) purifying said compound of claim 2.

37. A method for synthesizing a compound of claim 2, comprising the steps of:
(a) reacting a first reactant of formula (V): 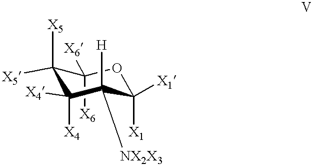
with a second reactant,
wherein X₁ and X₁′ are independently selected from the group consisting of hydrogen, —OCH₃, -alkyl, —O— alkyl, —O—C(O)-alkyl, —O—CH₂—CH₂ (O—C (O) —R₆) —CH₂ (O—C (O) —R₇), —O—CH₂—CH₂ (OR₆) —CH₂ (OR₇) , —O—CH₂—CH₂ (R₆) —CH₂ (R₇), —O—(CH₂)ₘ-cholesterol, —O—(CH₂),—N(R₈)₃, —NH₂, —N⁺(CH₃) ₃, and —(CH₂)ₙ—N(R₉)₃, wherein R₆, R₇, R₈, and R₉are independently selected from the group consisting of hydrogen, methyl, and alkyl, and wherein m is selected from the group consisting of 0, 1, 2, 3, 4, and 5, and wherein n is selected from the group consisting of 1, 2, 3, 4, and 5; and wherein X₂ and X₃ are independently selected from the group consisting of hydrogen and a protecting group, and X₄, X₄′, X₅, X₅, X₅′, X₆, and X₆′ are independently selected from the group consisting of hydrogen, —OH, and —O— alkyl;
wherein said second reactant is selected from the group consisting of ClCH₃, Cl-alkyl, Cl—CH₂—CH₂ (O—C (O) —R₆) —CH₂ (O—C (O) —R₇), Cl—CH₂—CH₂ (OR₆) —CH₂ (OR₇) , Cl—CH₂—CH₂ (R₆) —CH₂ (R₇), Cl—(CH₂)ₘ-cholesterol, and Cl—(CH₂)ₙ—N (R₈)₃, wherein R₆, R₇, R₈, and R₉are independently selected from the group consisting of hydrogen, methyl, and alkyl, and wherein m is selected from the group consisting of 0, 1, 2, 3, 4, and 5, and wherein n is selected from the group consisting of 1, 2, 3, 4, and 5;
(b) reacting the product of step (a) with a reducing agent and a catalyst; and
(c) purifying said compound of claim 2.

38. The method of claims 36 and 37, wherein said protecting group is an N-phthalimido moiety.

39. The method of claims 36 and 37, wherein said reducing agent is selected from the group consisting of H₂NNH₂, H₂, and NABH₄.

40. The method of claim 37, wherein said catalyst is palladium.
